(19) United States
(12) Patent Application Publication
(10) Pub. No.: US 2011/0136843 A1
(43) Pub. Date: Jun. 9, 2011

(54) Novel Combination of Compounds to be Used in the Treatment of Airway Diseases, Especially Chronic Obstructive Pulmonary Disease (COPD) and Asthma

(76) Inventors: Tomas Eriksson, Lund (SE); Johan Hansson, Lund (SE); Marguerite Mensonides-Harsema, Hamburg (DE); John Mo, Lund (SE)

(21) Appl. No.: 12/527,760

(22) PCT Filed: Feb. 21, 2008
(86) PCT No.: PCT/SE08/50203

§ 371 Date: Dec. 21, 2010

Related U.S. Application Data
(60) Provisional application No. 60/891,245, filed on Feb. 23, 2007.

(51) Int. Cl. A61K 31/4468; C07D 211/58; A61K 31/438; A61P 11/00; A61P 11/06

(52) U.S. Cl. 514/278; 514/291; 546/223

(57) ABSTRACT

The present invention relates to a combination of (a) a chemokine receptor 1 (CCR1) antagonist and (b) a muscarinic antagonist. The invention further relates to pharmaceutical compositions comprising said combination and to methods of treatment of airway diseases, such as chronic obstructive pulmonary disease (COPD) and asthma in mammals by administrating said combination. The invention further relates to a kit comprising the combination and use of said kit in treatment of airway diseases.

### THE FIELD OF THE INVENTION

The present invention relates to a combination of (a) a chemokine receptor 1 (CCR1) antagonist and (b) a muscarinic antagonist. The invention further relates to pharmaceutical compositions comprising said combination and to methods of treatment of airway diseases, such as chronic obstructive pulmonary disease (COPD) and asthma in mammals by administrating said combination. The invention further relates to a kit comprising the combination and use of said kit in treatment of airway diseases.

### BACKGROUND OF THE INVENTION

The essential function of the lungs requires a fragile structure with enormous exposure to the environment, including pollutants, microbes, allergens, and carcinogens. Host factors, resulting from interactions of lifestyle choices and genetic composition, influence the response to this exposure. Damage or infection to the lungs can give rise to a wide range of diseases of the respiratory system (or airway diseases). A number of these diseases are of great public health importance. Airway diseases include Acute Lung Injury, Acute Respiratory Distress Syndrome (ARDS), occupational lung disease, lung cancer, tuberculosis, fibrosis, pneumoconiosis, pneumonia, emphysema, Chronic Obstructive Pulmonary Disease (COPD) and asthma.

Among the most common airway diseases is asthma. Asthma is generally defined as an inflammatory disorder of the airways with clinical symptoms arising from intermittent airflow obstruction. It is characterised clinically by paroxysms of wheezing, dyspnea and cough. It is a chronic disabling disorder that appears to be increasing in prevalence and severity. It is estimated that 15% of children and 5% of adults in the population of developed countries suffer from asthma. Therapy should therefore be aimed at controlling symptoms so that normal life is possible and at the same time provide basis for treating the underlying inflammation.

COPD is a term which refers to a large group of lung diseases which can interfere with normal breathing. Current clinical guidelines define COPD as a disease state characterized by airflow limitation that is not fully reversible. The airflow limitation is usually both progressive and associated with an abnormal inflammatory response of the lungs to noxious particles and gases. The most important contributory source of such particles and gases, at least in the western world, is tobacco smoke. COPD patients have a variety of symptoms, including cough, shortness of breath, and excessive production of sputum; such symptoms arise from dysfunction of a number of cellular compartments, including neutrophils, macrophages, and epithelial cells. The two most important conditions covered by COPD are chronic bronchitis and emphysema.

Chronic bronchitis is a long-standing inflammation of the bronchi which causes increased production of mucous and other changes. The patients' symptoms are cough and expectoration of sputum. Chronic bronchitis can lead to more frequent and severe respiratory infections, narrowing and plugging of the bronchi, difficult breathing and disability.

Emphysema is a chronic lung disease which affects the alveoli and/or the ends of the smallest bronchi. The lung loses its elasticity and therefore these areas of the lungs become enlarged. These enlarged areas trap stale air and do not effectively exchange it with fresh air. This results in difficult breathing and may result in insufficient oxygen being delivered to the blood. The predominant symptom in patients with emphysema is shortness of breath.

WO01/98273, WO03/051839 and WO 04/005295 describe compounds having activity as pharmaceuticals, in particular as modulators of chemokine receptor (especially MIP-1α chemokine receptor), salts thereof and pharmaceutical compositions, and their potential use in treating various diseases.

The MIP-1α chemokine receptor CCR1 (chemokine receptor 1) is highly expressed in tissues affected in different autoimmune, inflammatory, proliferative, hyperproliferative and immunologically mediated diseases e.g. asthma and chronic obstructive pulmonary disease. Moreover, inflammatory cells (e.g. neutrophils and monocytes/macrophages) contribute to the pathogenesis of airway diseases such as COPD by secretion of proteolytic enzymes, oxidants and pharmacologic mediators. These cells are dependent on the function of CCR1 for recruitment and activation in lung tissues.

The muscarinic receptors M1, M2 and M3 are expressed in human lungs, M2 and M3 dominating in the airways and M1 found only in smaller peripheral airways. Most cell types in airways and lung including inflammatory cells express muscarinic receptors. Acetylcholine (ACh) being the classical neurotransmitter of the parasympattic nervous system is the main endougenous ligand binding to the muscarinic receptors. M3 receptors are expressed on airway smooth muscle cells and mediate bronchoconstriction leading to airway narrowing. M1 receptors facilitate cholinergic neurotransmission and enhance airway bronchoconstriction. The M2 receptor acts as a feedback autoreceptor inhibiting the release of ACh at the nerve endings. In airway smooth muscle cells activation of M2 receptors augment ACh-triggered smooth muscle contraction initiated by activation of M3 receptors, leading to bronchoconstriction and an overall reduced lung functional capacity being a hallmark of COPD.

M3 signalling mediates smooth muscle cell proliferation and accordingly contributes to the remodelling process in chronic inflammatory airways. M3 signalling enhances mucus production from airway goblet cells, contributing to plugging of small airways, leading to cough (bronchitis) and reduced lung function in COPD patients. Muscarinic agonists, such as ACh, act on airway tracheal epithelial cells and increase cell proliferation, release of inflammatory mediators from epithelial and inflammatory cells, which results in increased chemotactic activity of neutrophils and macrophages.

Treatment of COPD patients with inhaled M1 and M3 selective muscarinic antagonists targets cholinergic bronchoconstriction by opening narrowed airways resulting in sustained improvement in lung function, reduced mucus production, reduced exacerbation frequency, less activity-induced breathlessness, improved exercise endurance and an overall quality of life (QOL) improvement for these patients.

The present invention relates to a combination of a CCR1 antagonist with a muscarinic antagonist.

It is contemplated that the combination of the present invention has a beneficial therapeutic effect in the treatment of airway diseases. For example, the combination according to the invention is considered to be particularly effective in reducing inflammatory cell influx into the lung. The beneficial effect may be observed when the two active substances are administered simultaneously (either in a single pharmaceutical composition or in separate compositions), or sequentially or separately.

### DESCRIPTION OF THE FIGURES

FIGS. 1 to 4 show the results of a cell influx experiment in LPS-challenged rats using a combination of the present invention.

FIG. 5 shows the XRPD of the hemifumarate salt of N-{5-chloro-2-[((2S)-3-{[1-(4-chlorobenzyl)piperidin-4-yl]amino}-2-hydroxy-2-methylpropyl)oxy]-4-hydroxyphenyl}acetamide.

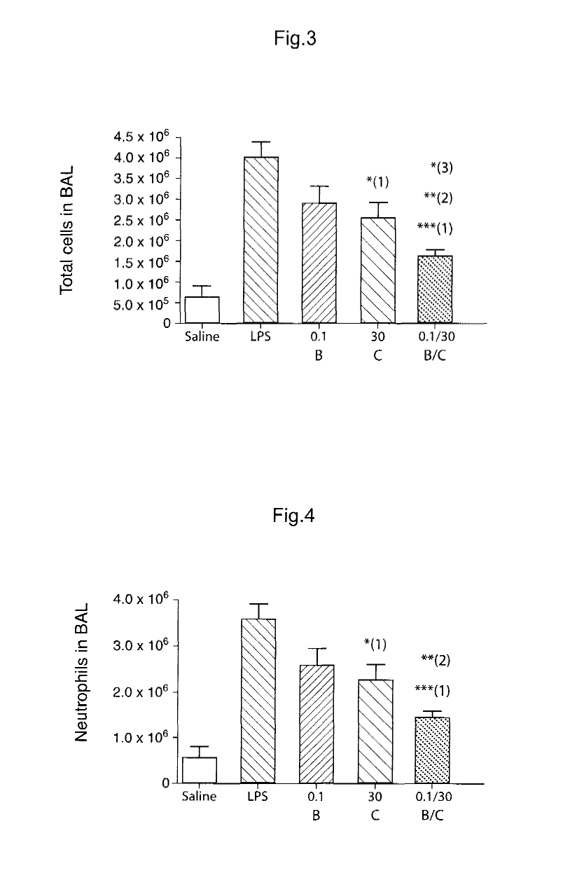

FIG. 6 shows the XRPD of the sulphate salt of N-{5-chloro-2-[((2S)-3-{[1-(4-chlorobenzyl)piperidin-4-yl]amino}-2-hydroxy-2-methylpropyl)oxy]-4-hydroxyphenyl}acetamide.

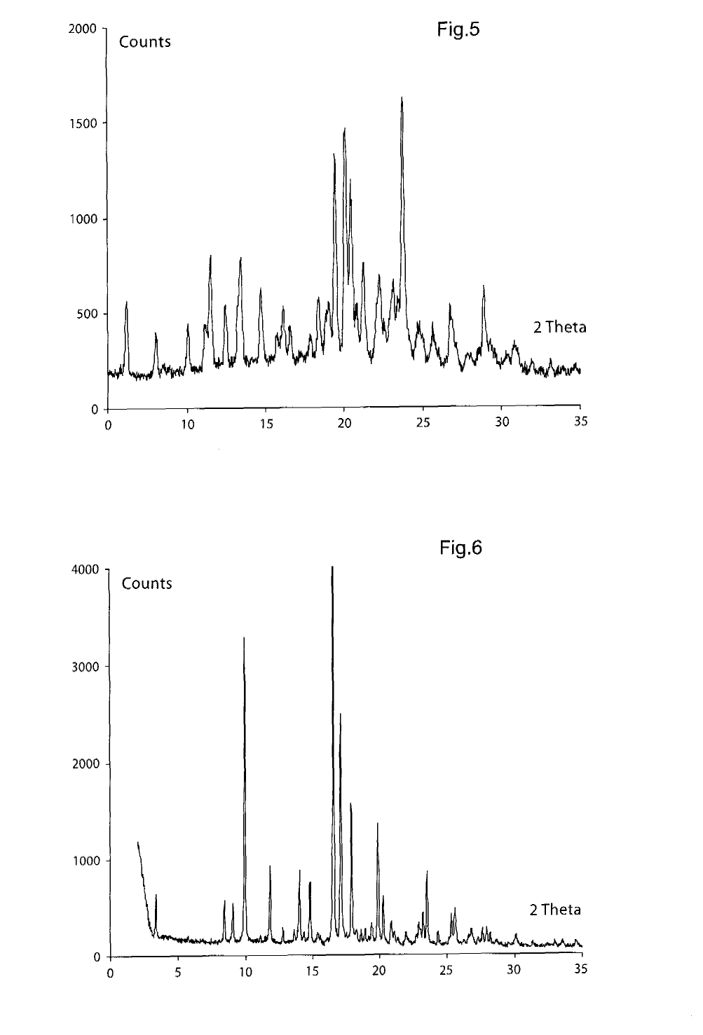

### DETAILED DESCRIPTION OF THE INVENTION

Thus, according to the present invention, there is provided a pharmaceutical product comprising, in combination,

(a1) a first active ingredient, which is a compound of general formula

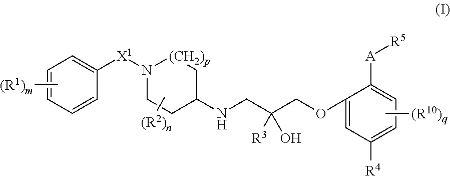

wherein:
m is 0, 1 or 2;
R¹ is halogen, C₁₋₃haloalkyl or cyano;
X¹ is —CH₂— or —C(O)—;
n is 0, 1 or 2;
p is 0, 1 or 2;
R² is C₁₋₆cycloalkyl; or
R² forms a bicyclic ring together with the ring it is attached to;
R³ is hydrogen or C₁₋₄alkyl;
R⁴ is hydrogen, halogen, hydroxyl, C₁₋₆hydroxyalkyl, optionally substituted by one substituent independently selected from halogen, cyano, amino (—NH₂), amido (—CONH₂), hydroxyl, oxo (═O), C₁₋₆haloalkyl, carboxyl, C₁₋₆ alkoxy, C₁₋₆alkoxycarbonyl, C₁₋₆alkylcarbonylamino and a 3 to 6-membered saturated or unsaturated ring, optionally comprising one or more heteroatom selected from nitrogen, oxygen and sulphur, and optionally further comprising a bridging group, the ring being optionally substituted with one or more substituent independently selected from halogen, hydroxyl, oxo (═O), C₁₋₆alkyl, C₁₋₆hydroxyalkyl and C₁₋₆haloalkyl;
A is a bond or C₁₋₆haloalkyl;
R⁵ is hydrogen, hydroxyl, —NHC(O)R⁶, —NHS(O)₂R⁶, —C(O)NR⁷R⁸, —COOR⁹ or SO₃R⁹;
R⁶ is hydrogen, C₁₋₆alkyl or a 3 to 6-membered saturated or unsaturated ring, optionally comprising one or more heteroatom selected from nitrogen, oxygen and sulphur, and optionally further comprising a bridging group, the ring being optionally substituted with one or more substituent independently selected from halogen, hydroxyl, C₁₋₆alkyl, C₁₋₆ hydroxyalkyl and C₁₋₆haloalkyl, oxo (═O) and —OR⁹;
R⁷ and R⁸ each independently represent (i) hydrogen atom,
   (ii) a 3 to 6-membered saturated or unsaturated ring, optionally comprising one or more heteroatom selected from nitrogen, oxygen and sulphur, and optionally further comprising a bridging group, the ring being optionally substituted with one or more substituent independently selected from halogen, hydroxyl, oxo (═O), C₁₋₆alkyl, C₁₋₆hydroxyalkyl and C₁₋₆haloalkyl,
   (iii) a C₁₋₆alkyl group, optionally substituted by one or more substituent independently selected from halogen, amino (—NH₂), hydroxyl, oxo (═O), C₁₋₆haloalkyl, carboxyl, Cᵢ-6alkoxy, C₁₋₆alkoxycarbonyl, C₁₋₆alkylcarbonylamino and a 3 to 6-membered saturated or unsaturated ring, optionally comprising one or more heteroatom selected from nitrogen, oxygen and sulphur, and optionally further comprising a bridging group, the ring being optionally substituted with one or more substituent independently selected from halogen, hydroxyl, oxo (═O), C₁₋₆alkyl, C₁₋₆hydroxyalkyl and C₁₋₆haloalkyl, or
   (iv) C₁₋₆alkylsulphonyl, or
   (v) R⁷ and R⁸ together with the nitrogen atom to which they are attached form a 4 to 7-s membered saturated heterocyclic ring that optionally further comprises a ring nitrogen, oxygen or sulphur atom and that is optionally fused to a benzene ring to form a 8 to 11-membered ring system, the heterocyclic ring or ring system being optionally substituted with one or more substituent independently selected from halogen, hydroxyl, amido (—CONH₂), C₁₋₆ alkyl, C₁₋₆hydroxyalkyl, C₁₋₆alkoxy, C₁₋₆ alkoxycarbonyl, C₁₋₆haloalkyl, C₁₋₆alkylamino, di-C₁₋₆alkylamino, C₁₋₆ alkylcarbonyl, C₁₋₆ alkylcarbonylamino, C₁₋₆alkylaminocarbonyl, di-C₁₋₆alkylaminocarbonyl, phenyl, halophenyl and phenylcarbonyl;
R⁹ is hydrogen or C₁₋₆alkyl;
q is 0, 1 or 2;
R¹⁰ is halogen, hydroxyl, cyano, C₁₋₃haloalkyl or C₁₋₆alkoxy;
   or a pharmaceutically acceptable salt thereof;

### Or,

(a2) a first active ingredient, which is a compound of general formula

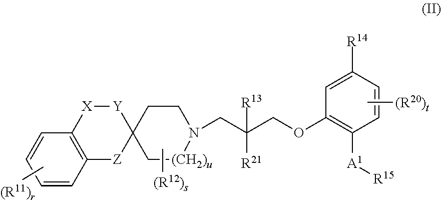

wherein:
r is 0, 1 or 2;
R¹¹ is halogen, cyano or C₁₋₆haloalkyl;
X, Y and Z is a bond, —O—, —NH—, CH₂— or —C(O)—, provided that only one of X, Y and Z is a bond, and provided that X and Y are not simultaneously —O— or —C(O)—;
s is 0, 1 or 2;
R¹² is C₁₋₆cycloalkyl;
u is 0 or 1;
R²¹ is hydrogen, hydroxyl or NH₂;
R¹³ is hydrogen or C₁₋₆alkyl;
A¹ is a bond or C₁₋₃alkyl;
R¹⁵ is hydrogen, hydroxyl, —NHC(O)R¹⁶, —NHS(O)₂R¹⁶, —C(O)NR¹⁷R¹⁸, —COOR¹⁹ or SO₃R¹⁹;
R¹⁴ is hydrogen, halogen, hydroxyl, OC(CH₃)₂COOH, C₁₋₆hydroxyalkyl optionally substituted by one or more substituent independently selected from halogen, cyano, amino (—NH₂), amido (—CONH₂), hydroxyl, oxo (═O), C₁₋₆haloalkyl, carboxyl, C₁₋₆alkoxy, C₁₋₆alkoxycarbonyl, C₁₋₆alkylcarbonylamino and a 3 to 6-membered saturated or unsaturated ring, optionally comprising one or more heteroatom independently selected from nitrogen, oxygen and sulphur, and optionally further comprising a bridging group, the ring being optionally substituted with one or more substituent independently selected from halogen, hydroxyl, oxo (═O), C₁₋₆alkyl, C₁₋₆hydroxyalkyl and C₁₋₆haloalkyl;
t is 0, 1 or 2;
R¹⁶ is hydrogen, C₁₋₃alkyl, NR¹⁷R¹⁸ or OR¹⁹;
R¹⁷ and R¹⁸ are independently selected from hydrogen, C₁₋₆alkyl and C₃₋₇cycloalkyl, or
R¹⁷ and R¹⁸ together with the nitrogen atom to which they are attached form a 4 to 7-membered heterocyclic ring, which is optionally substituted with on or more hydroxyl groups;
R¹⁹ is a hydrogen or C₁₋₃alkyl group; and
R²⁰ is halogen, cyano, C₁₋₃alkoxy or C₁₋₃haloalkyl,
   or a pharmaceutically acceptable salt thereof;
   and
   (b) a second active ingredient, which is a muscarinic antagonist, or a pharmaceutically acceptable salt thereof,
   provided the muscarinic antagonist is not selected from
a [2-((S)-Cyclohexyl-hydroxy-phenyl-methyl)-oxazol-5-ylmethyl]-dimethyl-(3-phenoxy-propyl)-ammonium salt,
a [2-((R)-Cyclohexyl-hydroxy-phenyl-methyl)-oxazol-5-ylmethyl]-dimethyl-(3-phenoxy-propyl)-ammonium salt,
a [2-((R)-Cyclohexyl-hydroxy-phenyl-methyl)-oxazol-5-ylmethyl]-dimethyl-(2-phenethyloxy-ethyl)-ammonium salt,
a [2-((R)-Cyclohexyl-hydroxy-phenyl-methyl)-oxazol-5-ylmethyl]-[3-(3,4-dichloro-phenoxy)-propyl]-dimethyl-ammonium salt,
a [2-((R)-Cyclohexyl-hydroxy-phenyl-methyl)-oxazol-5-ylmethyl]-[2-(3,4-dichloro-benzyloxy)-ethyl]-dimethyl-ammonium salt, or
a [2-(4-Chloro-benzyloxy)-ethyl]-[2-((R)-Cyclohexyl-hydroxy-phenyl-methyl)-oxazol-5-ylmethyl]-dimethyl-ammonium salt.

In another embodiment there is provided a pharmaceutical product comprising, in combination,

(a1) a first active ingredient, which is a compound of general formula

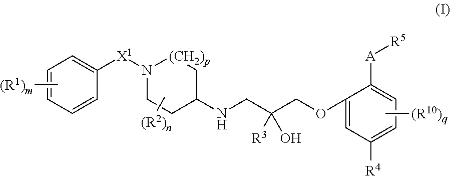

wherein:
m is 0 or 1; R¹ is halogen; X¹ is —CH₂—; n is 0; p is 0 or 1; R³ is C₁₋₄alkyl; R⁴ is hydroxyl; A is a bond; R⁵ is —NHC(O)R⁶; R⁶ is C₁₋₆alkyl and q is 0; or a pharmaceutically is acceptable salt thereof;
   or,
   (a2) a first active ingredient, which is a compound of general formula

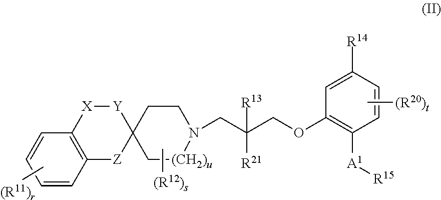

wherein:
r is 0 or 1; R¹¹ is halogen; X, Y and Z is a bond, —O— or CH₂—, provided that only one of X,
Y and Z is a bond, and provided that X and Y are not simultaneously —O—; s is 0; u is 0 or 1;
R²¹ is hydroxyl; R¹³ is hydrogen; A¹ is a bond; R¹⁵ is —C(O)NR¹⁷R¹⁸; R¹⁴ is OC(CH₃)₂COOH; t is 0 or 1; R¹⁷ and R¹⁸ are independently selected from hydrogen and C₁₋₆alkyl; and R²⁰ is halogen, or a pharmaceutically acceptable salt thereof; and
   (b) a second active ingredient, which is a muscarinic antagonist, or a pharmaceutically acceptable salt thereof,
   provided the muscarinic antagonist is not selected from a
a [2-((S)-Cyclohexyl-hydroxy-phenyl-methyl)-oxazol-5-ylmethyl]-dimethyl-(3-phenoxy-propyl)-ammonium salt,
a [2-((R)-Cyclohexyl-hydroxy-phenyl-methyl)-oxazol-5-ylmethyl]-dimethyl-(3-phenoxy-propyl)-ammonium salt,
a [2-((R)-Cyclohexyl-hydroxy-phenyl-methyl)-oxazol-5-ylmethyl]-dimethyl-(2-phenethyloxy-ethyl)-ammonium salt,
a [2-((R)-Cyclohexyl-hydroxy-phenyl-methyl)-oxazol-5-ylmethyl]-[3-(3,4-dichloro-phenoxy)-propyl]-dimethyl-ammonium salt,
a [2-((R)-Cyclohexyl-hydroxy-phenyl-methyl)-oxazol-5-ylmethyl]-[2-(3,4-dichloro-benzyloxy)-ethyl]-dimethyl-ammonium salt, or
a [2-(4-Chloro-benzyloxy)-ethyl]-[2-((R)-Cyclohexyl-hydroxy-phenyl-methyl)-oxazol-5-ylmethyl]-dimethyl-ammonium salt.

In one embodiment there is provided a pharmaceutical product as defined above comprising:

(a) a first active ingredient, which is a compound of general formula (I) or (II) as defined above, and
(b) a second active ingredient, which is tiotropium or a pharmaceutically acceptable salt thereof.

In another embodiment the second active ingredient is tiotropium bromide.

In one embodiment of the invention where the first active ingredient is a compound of formula (I) m is 1 and R¹ is a halogen atom. In one embodiment R¹ is chlorine or fluorine.

In a further embodiment, where the first active ingredient is a compound of formula (I), m is 1 and R¹ is chlorine in the 4-position of the benzene ring relative to the carbon atom to which the CH₂ linking group is attached.

In another embodiment, where the first active ingredient is a compound of formula (I), X¹ is a —CH₂— or a —C(O)—. In one embodiment X¹ is —CH₂—. In a further embodiment, X¹ is —C(O)—.

In a further embodiment, where the first active ingredient is a compound of formula (I), the integer n is 0, 1 or 2. In one embodiment n is 0. In another embodiment n is 1 or 2.

In another embodiment, where the first active ingredient is a compound of formula (I), R² is C₁₋₆alkyl. In one embodiment n is 2 and R² is methyl.

In one embodiment, where the first active ingredient is a compound of formula (I) p is 1.

In yet a further embodiment, where the first active ingredient is a compound of formula (I),

R³ is methyl, ethyl, n-propyl, isopropyl, n-butyl, isobutyl or tert-butyl. In one embodiment R³ is methyl.

In yet a further embodiment, where the first active ingredient is a compound of formula (I) A is a bond.

In one embodiment, where the first active ingredient is a compound of formula (I) R⁵ is —NHC(O)R⁶ and R⁶ is methyl, ethyl, n-propyl, isopropyl, n-butyl, isobutyl or tert-butyl.

In another embodiment R⁵ is —NHC(O)R⁶ and R⁶ is methyl.

In a further embodiment, where the first active ingredient is a compound of formula (I) R⁴ is hydroxyl.

In yet another embodiment of the present invention, where the first active ingredient is a compound of formula (I), the integer q is 0 or 1. In one embodiment q is 0. In yet another embodiment q is 1.

In a further embodiment of the present invention, where the first active ingredient is a compound of formula (I) R¹⁰ is a halogen, such as chlorine and fluorine. In one embodiment q is 1 and R¹⁰ is chlorine.

In another embodiment where the first active ingredient is a compound of formula (I) m is 1, R¹ is chloride, X¹ is —CH₂—, n is 0 and p is 1.

In a further embodiment where the first active ingredient is a compound of formula (I) R³ is methyl, A is a bond, R⁵ is —NHC(O)R⁶ and R⁶ is methyl, R⁴ is hydroxyl and q is 0.

In one embodiment of the invention where the first active ingredient is a compound of formula (II) m is 1 and R¹¹ is a halogen atom. In one embodiment R¹¹ is chlorine.

In another embodiment, where the first active ingredient is a compound of formula (II) X, Y and Z are a bond, —O—, —NH—, CH₂— or —C(O)—, provided that only one of X, Y and Z is a bond, and provided that X and Y are not simultaneously —O— or —C(O)—.

In one embodiment X is —O—, Y is a bond and Z is CH₂. In yet another embodiment X is a bond, Y is —NH—, and Z is —C(O). In yet another embodiment, X is —CH₂, Y is —O— and Z is a bond.

In a further embodiment, where the first active ingredient is a compound of formula (II), the integer s is 0, 1 or 2. In one embodiment s is 0. In another embodiment s is 1 or 2.

In another embodiment, where the first active ingredient is a compound of formula (II), R¹² is C₁₋₆alkyl. In one embodiment of the present invention, s is 2 and R¹² is methyl.

In one embodiment, where the first active ingredient is a compound of formula (II) u is 1.

In yet a further embodiment of the present invention, where the first active ingredient is a compound of formula (II), R¹³ is hydrogen, methyl, ethyl, n-propyl, isopropyl, n-butyl, isobutyl or tert-butyl. In another embodiment R¹³ is hydrogen. In one embodiment, R¹³ is methyl.

In yet another embodiment, where the first active ingredient is a compound of formula (II) R²¹ is hydrogen, hydroxyl or amino group. In one embodiment R²¹ is hydrogen. In yet another embodiment R²¹ is hydroxyl. In one embodiment R²¹ is an NH₂.

In another embodiment where the first active ingredient is a compound of formula (II) m is 1, R¹¹ is chloride, X is —O—, Y is a bond and Z is CH₂, s is 0, u is 1, R¹³ is hydrogen and R²¹ is hydroxyl.

In one embodiment, where the first active ingredient is a compound of formula (II), R¹⁴ is hydrogen, halogen, hydroxyl or C₁₋₆hydroxyalkyl, optionally substituted with halogen, cyano, hydroxyl, carboxyl or amido. In one embodiment R¹⁴ is hydrogen. In another embodiment R¹⁴ is halogen such as fluorine. In another embodiment R¹⁴ is hydroxyl. In yet another embodiment R¹⁴ is —OCH₂COOH. In yet a further embodiment R¹⁴ is —OC(CH₃)₂COOH.

In another embodiment of the present invention, where the first active ingredient is a compound of formula (II) R¹⁴ is selected from —OCH₂CF₃, —OCH₂CH₂CF₃, —OCH₂CHF₂ or —OCH₂CN.

In a further embodiment of the present invention, where the first active ingredient is a compound of formula (II) R²⁰ is a halogen, such as chlorine and fluorine. In one embodiment t is 1 and R² is chlorine.

In one embodiment of the present invention, where the first active ingredient is a compound of formula (II) A¹ is a bond or methyl, ethyl, n-propyl or isopropyl. In one embodiment A¹ is a bond. In another embodiment A¹ is methyl or ethyl.

In a further embodiment of the present invention, where the first active ingredient is a compound of formula (II) R¹⁵ is —NHC(O)R¹⁶, —NHS(O)₂R¹⁶, —C(O)NR¹⁷R¹⁸, and suitable R¹⁶, R¹⁷ and R¹⁸ are independently selected from hydrogen, methyl, ethyl, n-propyl or isopropyl. In one embodiment R¹⁵ is —C(O)NR¹⁷R¹⁸ and R¹⁷ is hydrogen and R¹⁸ is methyl.

In one embodiment of the present invention, where the first active ingredient is a compound of formula (II) R¹⁶ is —NR¹⁷R¹⁸, and R¹⁷ and R¹⁸ are independently selected from hydrogen, methyl, ethyl, n-propyl or isopropyl. In another embodiment R¹⁸ is methyl. In another embodiment A¹ is a bond, R¹⁶ is —NR¹⁷R¹⁸, R¹⁷ is hydrogen and R¹⁸ is methyl. In one embodiment A¹ is a bond, R¹⁶ is —NR¹⁷R¹⁸ and R¹⁷ and R¹⁸ are both hydrogen, methyl, ethyl, n-propyl or isopropyl. In another embodiment R¹⁷ and R¹⁸ are both methyl.

In a further embodiment of the present invention, where the first active ingredient is a compound of formula (II) A¹ is a bond, R¹⁵ is —C(O)NR¹⁷R¹⁸ and R¹⁷ is hydrogen and R¹⁸ is methyl, t is 1, R²⁰ is chlorine, R¹⁴ is —OC(CH₃)₂COOH.

In yet another embodiment of the present invention, where the first active ingredient is a compound of formula (II) A¹ is a bond, R¹⁵ is —NHC(O)R¹⁶, R¹⁶ is —NR¹⁷R¹⁸ and R¹⁷ and R¹⁸ together with the nitrogen atom to which they are attached form a 4 to 7-membered heterocyclic ring, which is optionally substituted with one or more hydroxyl groups.

In a further embodiment of the present invention, where the first active ingredient is a compound of formula (II) A¹ is a bond, R¹⁵ is —C(O)NR¹⁷R¹⁸ and R¹⁷ and R¹⁸ together with the nitrogen atom to which they are attached form a 4 to 7-membered heterocyclic ring, which is optionally substituted with one or more hydroxyl groups. In one embodiment heterocyclic groups for R¹⁷ and R¹⁸ and the nitrogen atom to which they are attached include azetininyl, pyrrolidinyl, piperadinyl and pyrrolidinyl.

In a further embodiment of the present invention, where the first active ingredient is a compound of formula (II) A¹ is methyl or ethyl and R¹⁵ is OH. In another embodiment of the present invention A¹ is methyl or ethyl and R¹⁵ is a group —COOR¹⁹ or —SO₃R¹⁹, where suitable R¹⁹ substituents are independently selected from hydrogen or C₁₋₃alkyl, such as methyl and ethyl.

For the avoidance of doubt, the present invention relates to a pharmaceutical product whereby the muscarinic antagonist is combined with any compound falling within the scope of compounds of formula (I) or (II) as defined above.

For the avoidance of doubt it is to be understood that where in this specification a group is qualified by ‘hereinbefore defined’, ‘defined hereinbefore’ or ‘defined above’ the said group encompasses the first occurring and broadest definition as well as each and all of the other definitions for that group.

For the avoidance of doubt it is to be understood that in this specification ‘C₁₋₆’ means a carbon group having 1, 2, 3, 4, 5 or 6 carbon atoms.

In this specification, unless stated otherwise, the term “alkyl” includes both straight and branched chain alkyl groups and may be, but are not limited to methyl, ethyl, n-propyl, i-propyl, n-butyl, i-butyl, s-butyl, t-butyl, n-pentyl, i-pentyl, neo-pentyl, n-hexyl or i-hexyl. The term C₁₋₄ alkyl having 1 to 4 carbon atoms and may be but are not limited to methyl, ethyl, n-propyl, i-propyl or tert-butyl.

The term “alkoxy”, unless stated otherwise, refers to radicals of the general formula —O—R, wherein R is selected from a hydrocarbon radical. The term “alkoxy” may include, but is not limited to methoxy, ethoxy, propoxy, isopropoxy, butoxy, t-butoxy, isobutoxy, cyclopropylmethoxy, allyloxy or propargyloxy.

In this specification, unless stated otherwise, the term “cycloalkyl” refers to an optionally substituted, partially or completely saturated monocyclic, bicyclic or bridged hydrocarbon ring system. The term “C₁₋₆cycloalkyl” may be, but is not limited to cyclopropyl, cyclobutyl, cyclopentyl or cyclohexyl.

In this specification, unless stated otherwise, the term “3 to 8-membered saturated or unsaturated ring, optionally comprising one or more heteroatom selected from nitrogen, oxygen and sulphur, or the term “4 to 7-membered heterocyclic ring” refers to a ringsystem having, in addition to carbon atoms, zero to three heteroatoms, including the oxidized form of nitrogen and sulfur and any quaternized form of a basic nitrogen, including, but not limited to cyclopropane, oxirane, cyclobutane, azetidine, cyclopentane, cyclohexane, benzyl, furane, thiophene, pyrrolidine, morpholine, piperidine, piperazine, pyrazine, azepane.

In this specification, unless stated otherwise, the term “bicyclic ring” refers to a ringsystem in which one (carbo)cycle is fused to another (carbo)cycle. The term “a 8 to 11-membered ring system” refers to a hydrocarbon moiety comprising one to three fused rings, optionally having 6, 10 or 14 π atoms shared in a cyclic array and having, in addition to carbon atoms, zero to five heteroatoms. Fused ringsystems may include, but are not limited to, 8-azabicyclo[3.2.1]octane, 3-azabicyclo[3.2.1]octane, 2-azabicyclo[2.2.2]octane, indole, indoline, benzofuran, benzothiophene, naphtalene, chroman, quinazoline, phenoxazine, azulene, adamantane, anthracene or phenoxazine.

In this specification, unless stated otherwise, the terms “halo” and “halogen” may be fluorine, iodine, chlorine or bromine.

In this specification, unless stated otherwise, the term “haloalkyl” means an alkyl group as defined above, which is substituted with halogen as defined above. The term “C₁-C₆haloalkyl” may include, but is not limited to fluoromethyl, difluoromethyl, trifluoromethyl, fluoroethyl, difluoroethyl or bromopropyl. The term “C₁₋₃haloalkylO” may include, but is not limited to fluoromethoxy, difluoromethoxy, trifluoromethoxy, fluoroethoxy or difluoroethoxy. The term “halophenyl” may include, but is not limited to fluorophenyl, difluorophenyl, trifluorophenyl, chlorophenyl, dichlorophenyl or trichlorophenyl.

In this specification, unless stated otherwise, the term “alkylcarbonyl” or “alkoxycarbonyl” may include, but is not limited to an alkyl or alkoxy group as defined above, which is substituted with COOH.

In this specification, unless stated otherwise, the term “alkylcarbonylamino” may include, but is not limited to an alkyl group as defined above, which is substituted with NHCOOH. In this specification, unless stated otherwise, the term “hydroxyalkyl” may include, but is not limited to an alkyl group as defined above, which is substituted with one or more hydroxyl groups.

It will be appreciated that throughout the specification, the number and nature of substituents on rings in the compounds of the invention will be selected so as to avoid sterically undesirable combinations.

In another embodiment of the present invention, the compound of formula (I) is selected from
N-(2{(2S)-3[{(3R)-1-[(4-chlorophenyl)methyl]-3-pyrrolidinyl}amino]-2-hydroxypropoxy}-4-fluorophenyl)acetamide;
N-(2{(2S)-3[{(3S)-1-[(4-chlorophenyl)methyl]-3-pyrrolidinyl}amino]-2-hydroxypropoxy}-4-fluorophenyl)acetamide;
N-(2-{(2S)-3-[1-{(4-chlorobenzoyl)-4-piperidinyl}amino]-2-hydroxypropoxy}-4-hydroxyphenyl)acetamide;
(2-{[(2S)-3-{[(2R,5S)-1-(4-chlorobenzyl)-2,5-dimethylpiperidin-4-yl]amino}-2-hydroxy-2-methylpropyl]oxy}-4-fluorophenyl)acetic acid;
(2-{[(2S)-3-{[(3S,4R)-1-(4-chlorobenzyl)-3-methylpiperidin-4-yl]amino}-2-hydroxy-2-methylpropyl]oxy}-4-fluorophenyl)acetic acid;
(2-{[(2S)-3-{[(3R,4R)-1-(4-chlorobenzyl)-3-methylpiperidin-4-yl]amino}-2-hydroxy-2-methylpropyl]oxy}-4-fluorophenyl)acetic acid;
(2-{[(2S)-3-{[(2R,4S,5S)-1-(4-chlorobenzyl)-2,5-dimethylpiperidin-4-yl]amino}-2-hydroxy-2-methylpropyl]oxy}-4-fluorophenyl)acetic acid;
(2-{[(2S)-3-{[(2R,4R,5S)-1-(4-chlorobenzyl)-2,5-dimethylpiperidin-4-yl]amino}-2-hydroxy-2-methylpropyl]oxy}-4-fluorophenyl)acetic acid;
(2-{[(2S)-3-{[(2S,4R,5R)-1-(4-chlorobenzyl)-2,5-dimethylpiperidin-4-yl]amino}-2-hydroxy-2-methylpropyl]oxy}-4-fluorophenyl)acetic acid;
(2-{[(2S)-3-{[(2S,4S,5R)-1-(4-chlorobenzyl)-2,5-dimethylpiperidin-4-yl]amino}-2-hydroxy-2-methylpropyl]oxy}-4-fluorophenyl)acetic acid;
Methyl (2-{[(2S)-3-{[1-(4-chlorobenzyl)piperidin-4-yl]amino}-2-hydroxypropyl]oxy}-4-fluorophenyl)propanoate;
N-[2-({2S}-3-[(1-[4-chlorobenzyl]-4-piperidinyl)amino]-2-hydroxypropoxy)-4-chlorophenyl acetamide;
N-[2-({2S}-3-[(1-[4-chlorobenzyl]-4-piperidinyl)amino]-2-hydroxy-2-methylpropoxy)-4-hydroxyphenyl]acetamide;
N-[2-({2S}-3-[(1-[4-chlorobenzyl]-4-piperidinyl)amino]-2-hydroxy-2-methylpropoxy)-4-fluorophenyl]acetamide;
N-[5-chloro-[2-({2S}-3-[(1-[4-chlorobenzyl]-4-piperidinyl)amino]-2-hydroxy-2-methylpropoxy)-4-hydroxyphenyl]acetamide;
N-[5-chloro-[2-({2S}-3-[(1-[4-chlorobenzyl]-4-piperidinyl)amino]-2-hydroxy-2-methylpropoxy)-4-hydroxyphenyl]propaneamide;
(2-{[(2S)-3-{[1-(4-chlorobenzyl)piperidin-4-yl]amino}-2-hydroxy-2-methylpropyl]oxy}-4-fluorophenyl)methanesulfonic acid;
Urea, N-5-chloro-(2-{(2S)-3-[1-{(4-chlorobenzyl)-4-piperidinyl}amino]-2-hydroxypropoxy}-4-hydroxyphenyl)-N′-cyclopropyl-;
Urea, N-(2-{(2S)-3-[1-{(4-chlorobenzyl)-4-piperidinyl}amino]-2-hydroxypropoxy}-phenyl)-N′-ethyl-;
(2S)-1-(2-ethylphenoxy)-3[(1-[4-chlorobenzyl]-4-piperidinyl)amino]propan-2-ol;
(2S)-1-[2-(-hydroxyethyl)phenoxy]-2-methyl-3[(1-[4-chlorobenzyl]-4-piperidinyl)amino]propan-2-ol;
2-({2S}-3-[(1-[4-chlorobenzyl]-4-piperidinyl)amino]-2-hydroxy-2-methylpropoxy)benzaldehyde;
2-({2S}-3-[(1-[4-chlorobenzyl]-4-piperidinyl)amino]-2-hydroxypropoxy)-N-cyclopropylbenzamide; and
Methyl 2-({2S}-3-[(1-[4-chlorobenzyl]-4-piperidinyl)amino]-2-hydroxypropoxy)-4-fluorobenzoate;
or a pharmaceutically acceptable salt thereof.

In another embodiment of the present invention, the compound of formula (II) is selected from
N-(2-{[(2S)-3-(5-chloro-1′H,3H-spiro[1-benzofuran-2,4′-piperidin]-1′-yl)-2-hydroxypropyl]oxy}-4-hydroxyphenyl)acetamide;
N-(2-{[(2S)-3-(5-chloro-1′H-spiro[1,3-benzodioxole-2,4′-piperidin]-1′-yl)-2-hydroxypropyl]oxy}-4-hydroxyphenyl)acetamide trifluoroacetate (salt);
2-{[(2S)-3-(5-chloro-1′H,3H-spiro[1-benzofuran-2,4′-piperidin]-1′-yl)-2-hydroxypropyl]oxy}-4-hydroxy-N-methylbenzamide trifluoroacetate (salt);
2-{[(2S)-3-(5-chloro-1′H,3H-spiro[1-benzofuran-2,4′-piperidin]-1′-yl)-2-hydroxypropyl]oxy}-4-hydroxybenzoic acid trifluoroacetate (salt);
N-(2-{[(2S)-3-(5-chloro-1′H,3H-spiro[2-benzofuran-1,4′-piperidin]-1′-yl)-2-hydroxypropyl]oxy}-4-hydroxyphenyl)acetamide trifluoroacetate (salt);
2-{[(2S)-3-(5-chloro-1′H,3H-spiro[2-benzofuran-1,4′-piperidin]-1′-yl)-2-hydroxypropyl]oxy}-4-hydroxy-N-methylbenzamide;
N-(2-{[(2S)-3-(5-fluoro-1′H,3H-spiro[1-benzofuran-2,4′-piperidin]-1′-yl)-2-hydroxypropyl]oxy}-4-hydroxyphenyl)acetamide;
2-{[(2S)-3-(5-fluoro-1′H,3H-spiro[1-benzofuran-2,4′-piperidin]-1′-yl)-2-hydroxypropyl]oxy}-4-hydroxy-N-methylbenzamide;
N-[2-({(2S)-3-[(2R)-5-chloro-1′H,3H-spiro[1-benzofuran-2,3′-pyrrolidin]-1′-yl]-2-hydroxypropyl}oxy)-4-hydroxyphenyl]acetamide;
N-(2-{[(2S)-3-(5-chloro-1′H,3H-spiro[1-benzofuran-2,4′-piperidin]-1′-yl)-2-hydroxypropyl]oxy}-4-hydroxyphenyl)urea trifluoroacetate (salt);
4-fluoro-2-{[(2S)-3-(5-fluoro-1′H,3H-spiro[1-benzofuran-2,4′-piperidin]-1′-yl)-2-hydroxypropyl]oxy}benzoic acid hydrochloride;
N-(2-{[(2S)-3-(5-chloro-1′H,3H-spiro[1-benzofuran-2,4′-piperidin]-1′-yl)-2-hydroxypropyl]oxy}-4-fluorophenyl)urea trifluoroacetate (salt);
N-(2-{[(2S)-2-amino-3-(5-fluoro-1′H,3H-spiro[1-benzofuran-2,4′-piperidin]-1′-yl)propyl]oxy}-4-hydroxyphenyl)acetamide bis(trifluoroacetate) (salt);
Benzaldehyde, 2-[(2S)-3-(5-chlorospiro[benzofuran-2(3H), 4′-piperidin]-1′-yl)-2-hydroxypropoxy]-;
Spiro[benzofuran-2(3H), 4′-piperidine]-1′-ethanol, 5-chloro-α-[[2-(2-hydroxyethyl)phenoxy]methyl]-, (αS)-;
Spiro[benzofuran-2(3H), 4′-piperidine]-1′-ethanol, 5-chloro-α-[[2-(hydroxymethyl)phenoxy]methyl]-, (αS)-;
N-(2-{[(2S)-3-(5-chloro-1′H,3H-spiro[1-benzofuran-2,4′-piperidin]-1′-yl)-2-hydroxypropyl]oxy}-5-chloro-4-hydroxyphenyl)acetamide;
2-Chloro-5-{[(2S)-3-(5-chloro-1′H,3H-spiro[1-benzofuran-2,4′-piperidin]-1′-yl)-2-hydroxypropyl]oxy}-(4-{acetylamino}phenoxy)acetic acid;
5-{[(2S)-3-(5-Chloro-1′H,3H-spiro[1-benzofuran-2,4′-piperidin]-1′-yl)-2-hydroxypropyl]oxy}-(4-{acetylamino}phenoxy)acetic acid;
{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}acetic acid;
2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid;
(2-Chloro-5-{[(2S)-3-(5-chloro-1′H,3H-spiro[1-benzofuran-2,4′-piperidin]-1′-yl)-2-hydroxypropyl]oxy}-4-{[(3S)-3-hydroxypyrrolidin-1-yl]carbonyl}phenoxy)acetic acid;
5-Chloro-2-{[(2S)-3-(5-chloro-1′H,3H-spiro[1-benzofuran-2,4′-piperidin]-1′-yl)-2-hydroxypropyl]oxy}-4-(cyanomethoxy)benzoic acid trifluoroacetate (salt);
2-{[(2S)-3-(5-chloro-1′H,3H-spiro[1-benzofuran-2,4′-piperidin]-1′-yl)-2-hydroxypropyl]oxy}-5-chloro-4-(2,2-difluoroethoxy)benzoic acid trifluoroacetate (salt);
5-Chloro-2-{[(2S)-3-(5-chloro-1′H,3H-spiro[1-benzofuran-2,4′-piperidin]-1′-yl)-2-hydroxypropyl]oxy}-4-(3,3,3-trifluoropropoxy)benzoic acid trifluoroacetate (salt);
N-(2-{3-[5-chloro-1′H,3H-spiro[1-benzofuran-2,4′-piperidin]-1′-yl]propoxy}phenyl)acetamide trifluoroacetate (salt);
Methyl 3-(2-{[(2S)-3-(5-chloro-1′H3H-spiro[1-benzofuran-2,4′-piperidin]-1′-yl)-2-hydroxypropyl]oxy}-4-fluorophenyl)propanoic acid trifluo acetic acid salt;
N-(2-{[(2S)-3-({spiro[indole-2-4′-piperidin]-3(1H)-one}-1′-yl)-2-hydroxypropyl]oxy}-4-hydroxyphenyl)acetamide; and
(2-{[(2S)-3-(5-Chloro-1′H,3H-spiro[1-benzofuran-2,4′-piperidin]-1′-yl)-2-hydroxypropyl]oxy}-4-fluorophenyl)methanesulfonic acid,
or a pharmaceutically acceptable salt, solvates or solvated salt thereof.

For the avoidance of doubt, the present invention relates to a pharmaceutical product whereby the muscarinic antagonist is combined with any one of the specific compounds of formula (I) or (II) as defined above.

In one embodiment there is provided a pharmaceutical product as defined above comprising:
(a) a first active ingredient, which is any one of N-{2-[((2S)-3-{[1-(4-chlorobenzyl)piperidin-4-yl]amino}-2-hydroxy-2-methylpropyl)oxy]-4-hydroxyphenyl}acetamide or N-{5-Chloro-2-[((2S)-3-{[1-(4-chlorobenzyl)piperidin-4-yl]amino}-2-hydroxy-2-methylpropyl)oxy]-4-hydroxyphenyl}acetamide or 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid or a pharmaceutically acceptable salt thereof, and
(b) a second active ingredient, which is a muscarinic antagonist or a pharmaceutically acceptable salt thereof, provided the muscarinic antagonist is not selected from
a [2-((S)-Cyclohexyl-hydroxy-phenyl-methyl)-oxazol-5-ylmethyl]-dimethyl-(3-phenoxy-propyl)-ammonium salt,
a [2-((R)-Cyclohexyl-hydroxy-phenyl-methyl)-oxazol-5-ylmethyl]-dimethyl-(3-phenoxy-propyl)-ammonium salt,
a [2-((R)-Cyclohexyl-hydroxy-phenyl-methyl)-oxazol-5-ylmethyl]-dimethyl-(2-phenethyloxy-ethyl)-ammonium salt,
a [2-((R)-Cyclohexyl-hydroxy-phenyl-methyl)-oxazol-5-ylmethyl]-[3-(3,4-dichloro-phenoxy)-propyl]-dimethyl-ammonium salt,
a [2-((R)-Cyclohexyl-hydroxy-phenyl-methyl)-oxazol-5-ylmethyl]-[2-(3,4-dichloro-benzyloxy)-ethyl]-dimethyl-ammonium salt, or
a [2-(4-Chloro-benzyloxy)-ethyl]-[2-((R)-Cyclohexyl-hydroxy-phenyl-methyl)-oxazol-5-ylmethyl]-dimethyl-ammonium salt.

In one embodiment there is provided a pharmaceutical product as defined above comprising:
(a) a first active ingredient, which is any one of N-{2-[((2S)-3-{[1-(4-chlorobenzyl)piperidin-4-yl]amino}-2-hydroxy-2-methylpropyl)oxy]-4-hydroxyphenyl}acetamide or N-{5-Chloro-2-[((2S)-3-{[1-(4-chlorobenzyl)piperidin-4-yl]amino}-2-hydroxy-2-methylpropyl)oxy]-4-hydroxyphenyl}acetamide or 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid or a pharmaceutically acceptable salt thereof, and
(b) a second active ingredient, which is tiotropium, or a pharmaceutically acceptable salt thereof.

In another embodiment second active ingredient is tiotropium bromide.

The CCR1 antagonists of the present invention have been named with the aid of computer software (ACDLabs 8.0/Name (IUPAC)).

The compound of formula (I) and (II) are capable of existing in stereoisomeric forms. It will be understood that the invention encompasses the use of all geometric and optical isomers of the compounds of formula (I) and (II) and mixtures thereof including racemates.

The use of tautomers and mixtures thereof also form an aspect of the present invention. In one embodiment the optical isomers are the (S)-enantiomers (i.e. compounds with the S configuration at the stereocentre with R³ and R¹³ or OH attached).

It will be appreciated that the compounds of formula (I) and (II) and salts thereof may exist as zwitterions. Thus, whilst the compounds are drawn and referred to in the neutral form, they may exist also in internal salt (zwitterionic) form. The representation of formula (I) and (II) and the compounds of the examples of the present invention covers both neutral and zwitterionic forms and mixtures thereof in all proportions.

The compounds of formula (I) and (II) may be used in the form of a pharmaceutically acceptable salt thereof, conceivably an acid addition salt such as a hydrochloride, hydrobromide, phosphate, sulfphate, acetate, ascorbate, benzoate, 2-fluorobenzoate, 2,6-difluorobenzoate, (hemi)fumarate, furoate, succinate, maleate, tartrate, citrate, oxalate, xinafoate, methanesulphonate, trifluoroacetate or p-toluenesulphonate. Pharmaceutically acceptable salts may also be formed together with metals such as calcium, magnesium, sodium, potassium or zinc or bases such as piperazine, 2-aminoethanol, choline, diethylamine or diethanol amine. Furthermore, the compounds of formula (I) and (II) may be used in the form of a pharmaceutically acceptable salt thereof, like an amino acid addition salt such as L-lysine, glycine, L-glutamine, L-asparagine or L-arganine A pharmaceutically acceptable salt also includes internal salt (zwitterionic) forms. Any reference to compounds of formula (I) and (II) or salts thereof also encompasses solvates of such compounds and solvates of such salts (e.g. hydrates) as well as cocrystals.

In another embodiment of the present invention, the compound of formula (I) is a salt of N-{2-[((2S)-3-{[1-(4-chlorobenzyl)piperidin-4-yl]amino}-2-hydroxy-2-methylpropyl)oxy]-4-hydroxyphenyl}acetamide or N-{5-Chloro-2-[((2S)-3-{[1-(4-chlorobenzyl)piperidin-4-yl]amino}-2-hydroxy-2-methylpropyl)oxy]-4-hydroxyphenyl}acetamide, for example hydrochloride, hydrobromide, phosphate, sulfphate, acetate, ascorbate, benzoate, fumarate, hemifumarate, furoate, succinate, maleate, tartrate, citrate, oxalate, xinafoate, methanesulphonate, p-toluenesulphonate, 2-fluorobenzoate or 2,6-difluorobenzoate salt.

One embodiment relates to the combination of the invention using the benzoate, furoate salts or hemifumarate salts of N-{2-[((2S)-3-{[1-(4-chlorobenzyl)piperidin-4-yl]amino}-2-hydroxy-2-methylpropyl)oxy]-4-hydroxyphenyl}acetamide, as described in WO2007/015666, WO2007/015667 and WO2007/015668.

Another embodiment relates to the hydrochloride, trifluoroacetate, p-toluensulfonate, sodium hydroxide, hemifumarate, furoate, benzoate, 2-fluorobenzoate or 2,6-difluorobenzoate salt of N-{5-Chloro-2-[((2S)-3-{[1-(4-chlorobenzyl)piperidin-4-yl]amino}-2-hydroxy-2-methylpropyl)oxy]-4-hydroxyphenyl}acetamide.

In yet another embodiment of the present invention, the compound of formula (II) is 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid. The preparation of this compound is described in WO2008/010765. In yet a further embodiment of the present invention, the compound of formula (II) is a hydrochloride, trifluoroacetate, p-toluensulfonate, sodium hydroxide, hemifumarate, furoate, benzoate, 2-fluorobenzoate or 2,6-difluorobenzoate salt of 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid as described in WO2008/010765.

In one embodiment of the invention, the compound of formula (I) is a hemifumarate salt of N-{2-[((2S)-3-{[1-(4-chlorobenzyl)piperidin-4-yl]amino}-2-hydroxy-2-methylpropyl)oxy]-4-hydroxyphenyl}acetamide which exhibits at least the following characteristic X-ray powder diffraction peaks (expressed in degrees 2θ):
(1) 6.3, 11.0 and 12.7, or
(2) 6.3, 10.7 and 12.7, or
(3) 6.3, 11.0, 12.7 and 15.9, or
(4) 6.3, 10.7, 11.0, 12.7, 13.9, 14.2 and 15.9, or
(5) 6.3, 10.7, 11.0, 12.7, 15.9, 17.7, 19.1, 19.7 and 25.5, or
(6) 6.3, 10.7, 11.0, 12.7, 13.9, 14.2, 15.9, 17.7, 19.1, 19.7, 19.9, 21.6 and 25.5.

In another embodiment of the invention, the compound of formula (I) is a furoate salt of N-{2-[((2S)-3-{[1-(4-chlorobenzyl)piperidin-4-yl]amino}-2-hydroxy-2-methylpropyl)oxy]-4-hydroxyphenyl}acetamide which exhibits at least the following characteristic X-ray powder diffraction peaks (expressed in degrees 2θ):
(1) 6.1, 10.7 and 19.3, or
(2) 6.1, 12.2 and 14.1, or
(3) 6.1, 10.7, 12.2, 14.1, 18.1 and 19.3, or
(4) 6.1, 10.7, 12.2, 14.1, 15.7, 18.1 and 19.3, or
(5) 6.1, 10.7, 12.2, 14.1, 15.1 and 19.3, or
(6) 6.1, 10.7, 12.2, 14.1, 15.1, 15.7, 18.1 and 19.3, or
(7) 6.1, 10.7, 12.2, 14.1, 15.1, 15.7, 18.1, 19.3, 21.2 and 24.6.

In yet another embodiment of the invention, the compound of formula (I) is a benzoate salt of N-{2-[((2S)-3-{[1-(4-chlorobenzyl)piperidin-4-yl]amino}-2-hydroxy-2-methylpropyl)oxy]-4-hydroxyphenyl}acetamide which exhibits at least the following characteristic X-ray powder diffraction peaks (expressed in degrees 2θ):
(1) 6.5, 9.3 and 10.5, or
(2) 6.5, 9.3, 17.6 and 17.8, or
(3) 6.5, 9.3, 10.5, 12.0 and 12.4, or
(4) 6.5, 9.3, 10.5, 12.0, 12.4, 13.0, 13.6, 15.5, 17.6 and 17.8, or
(5) 6.5, 13.0 and 20.2, or
(6) 6.5, 9.3, 10.5, 12.0, 12.4, 13.0, 13.6, 15.5, 17.6, 17.8 and 19.2, or
(7) 6.5, 9.3, 10.5, 12.0, 12.4, 13.0, 13.6, 15.5, 17.6, 17.8, 19.2, 20.2, 22.8 and 26.0, or
(8) 6.5, 9.3, 10.5, 12.0, 12.4, 13.0, 13.6, 15.5, 17.6, 17.8, 19.2, 20.2, 22.8, 24.2, 26.0 and 30.7.

The preparation of these polymorphic salts is described in WO2007/024182.

In a further embodiment of the invention, the compound of formula (I) is a hemifumarate salt of N-{5-chloro-2-[((2S)-3-{[1-(4-chlorobenzyl)piperidin-4-yl]amino}-2-hydroxy-2-methylpropyl)oxy]-4-hydroxyphenyl}acetamide which exhibits at least the following characteristic X-ray powder diffraction peaks (expressed in degrees 2θ):(BGJ 761-9701)
(1) 6.2, 14.7 and 20.5, or
(2) 8.0, 10.1 and 14.7, or
(3) 10.1, 12.4, 14.7 and 19.5, or
(4) 6.2, 10.1, 12.4, 13.4, 19.5 and 20.1, or
(5) 8.0, 10.1, 12.4, 14.7, 19.5, 20.1, 21.2 and 23.8, or
(6) 6.2, 8.0, 10.1, 11.5, 12.4, 13.4, 19.5, 20.1 and 21.2, or
(7) 6.2, 8.0, 11.5, 12.4, 13.4, 14.7, 20.1, 20.5, 21.2 and 23.8, or
(8) 6.2, 8.0, 10.1, 11.5, 12.4, 13.4, 14.7, 16.1, 20.5, 21.2 and 23.8

In another embodiment of the invention, the compound of formula (I) is a furoate salt of N-{5-chloro-2-[((2S)-3-{[1-(4-chlorobenzyl)piperidin-4-yl]amino}-2-hydroxy-2-methylpropyl)oxy]-4-hydroxyphenyl}acetamide which exhibits at least the following characteristic X-ray powder diffraction peaks (expressed in degrees 2θ):
(1) 6.7, 17.9 and 20.9 or
(2) 12.2, 13.3, 17.9 and 18.6 or
(3) 6.7, 12.2, 16.0, 17.3 and 27.0 or
(4) 6.7, 8.7, 12.2, 13.3, 16.0, 17.9 and 18.6 or
(5) 6.7, 12.2, 13.3, 16.0, 17.3, 17.9, 18.6, 20.9 and 27.0 or
(6) 6.7, 12.2, 13.3, 13.6, 15.5, 16.0, 17.3, 17.9, 18.6, 19.4, 20.9 and 27.3 or
(7) 6.7, 12.2, 13.3, 13.6, 15.5, 16.0, 17.3, 17.9, 18.6, 19.4, 20.9, 23.4 and 23.6 or
(8) 6.7, 12.2, 13.3, 13.6, 15.5, 16.0, 17.3, 17.9, 18.6, 19.4, 20.9, 23.4, 23.6, 27.0 and 27.3

In one embodiment of the invention, the compound of formula (I) is a benzoate salt of N-{5-chloro-2-[((2S)-3-{[1-(4-chlorobenzyl)piperidin-4-yl]amino}-2-hydroxy-2-methylpropyl)oxy]-4-hydroxyphenyl}acetamide which exhibits at least the following characteristic X-ray powder diffraction peaks (expressed in degrees 2θ):
(1) 5.6, 10.4 and 14.3 or
(2) 9.2, 12.9, 16.9, 19.5, and 25.4 or
(3) 5.6, 9.2, 11.3, 14.3, 16.9, 20.0 and 23.0 or
(4) 5.6, 11.3, 12.9, 18.0, 19.5, and 20.0, 23.0, 25.4 or,
(5) 5.6, 9.2, 10.4, 12.9, 14.3, 16.9, 18.0, 19.5, 20.0 and 25.4 or
(6) 5.6, 9.2, 10.4, 11.3, 12.9, 14.3, 16.9, 18.0, 19.5, 23.0 and 25.4 or
(7) 5.6, 9.2, 10.4, 11.3, 14.3, 16.9, 18.0, 19.5, 20.0, 23.0 and 25.4 or
(8) 5.6, 9.2, 10.4, 11.3, 12.9, 14.3, 16.9, 18.0, 19.5, 20.0, 23.0 and 25.4

In yet another embodiment of the invention, the compound of formula (I) is a 2-fluorobenzoate salt of N-{5-chloro-2-[((2S)-3-{[1-(4-chlorobenzyl)piperidin-4-yl]amino}-2-hydroxy-2-methylpropyl)oxy]-4-hydroxyphenyl}acetamide which exhibits at least the following characteristic X-ray powder diffraction peaks (expressed in degrees 2θ):
(1) 5.6, 9.2 and 11.2, or
(2) 5.6, 10.4 and 12.4, or
(3) 9.2, 10.4, 11.2, 12.4, 14.2 and 15.0 or
(4) 5.6, 10.4, 12.7, 13.6, 14.2, 15.0, 17.5 and 18.8 or
(5) 5.6, 9.2, 11.2, 12.4, 12.7, 13.6, 14.2, 15.0, 16.8, 17.7 and 19.1 or
(6) 9.2, 10.4, 11.2, 12.4, 13.6, 14.2, 15.0, 16.6, 18.8, 19.1 and 19.5 or
(7) 5.6, 9.2, 10.4, 12.4, 13.6, 14.2, 15.0, 16.8, 17.5, 18.8, 19.5 and 20.6 or
(8) 9.2, 10.4, 11.2, 12.4, 13.6, 14.2, 15.0, 16.6, 17.5, 17.7, 19.1, 19.5, 21.0, 22.6 and 25.2 or
(9) 5.6, 9.2, 10.4, 11.2, 12.4, 13.6, 14.2, 16.6, 16.8, 17.5, 18.8, 19.1, 19.5, 20.5, 21.0, and 22.6

In a further embodiment of the invention, the compound of formula (I) is a 2,6-difluorobenzoate salt of N-{5-chloro-2-[((2S)-3-{[1-(4-chlorobenzyl)piperidin-4-yl]amino}-2-hydroxy-2-methylpropyl)oxy]-4-hydroxyphenyl}acetamide which exhibits at least the following characteristic X-ray powder diffraction peaks (expressed in degrees 2θ):
(1) 5.6, 9.1 and 11.2, or
(2) 5.6, 10.3 and 12.6, or
(3) 9.1, 10.3, 11.2, 12.3, 14.9 and 16.7 or
(4) 5.6, 10.3, 12.6, 13.5, 14.2, 14.9, 17.4 and 19.0 or
(5) 5.6, 9.1, 11.2, 12.6, 13.5, 14.2, 16.7, 18.4, 18.7, 19.0 and 20.8 or
(6) 9.1, 10.3, 11.2, 12.3, 13.5, 14.2, 14.9, 16.7, 17.4, 18.7 and 19.0 or
(7) 5.6, 9.1, 10.3, 12.3, 13.5, 14.2, 14.9, 16.7, 17.4, 18.4, 19.5 and 20.3 or
(8) 9.1, 10.3, 11.2, 12.3, 13.5, 14.2, 14.9, 16.7, 17.4, 18.4, 18.7, 19.0, 20.3, 21.0 and 22.6 or
(9) 5.6, 9.1, 10.3, 11.2, 12.3, 13.5, 14.2, 16.7, 17.4, 18.7, 19.0, 19.5, 20.3, 20.8, and 23.4

The preparation of these polymorphic (di)fluorobenzoate salts is described in U.S. 60/889,759.

In another embodiment of the invention, the compound of formula (I) is a sulphate salt of N-{5-chloro-2-[((2S)-3-{[1-(4-chlorobenzyl)piperidin-4-yl]amino}-2-hydroxy-2-methylpropyl)oxy]-4-hydroxyphenyl}acetamide which exhibits at least the following characteristic X-ray powder diffraction peaks (expressed in degrees 2θ):
(1) 3.3, 9.9 and 19.8, or
(2) 8.4, 16.5 and 19.8, or
(3) 3.3, 9.9, 16.5, 17.9, 20.2 and 23.5, or
(4) 9.9, 16.5, 17.1, 17.9, 19.8, 20.2 and 23.5, or
(5) 3.3, 8.4, 9.9, 14.8, 17.1, 19.4, 19.8 and 20.2, or
(6) 9.0, 9.9, 11.8, 14.0, 14.8, 16.5, 19.8, 20.2 and 23.5, or
(7) 3.3, 8.4, 9.0, 9.9, 11.8, 12.7, 14.0, 17.1, 17.9, 19.4, 19.8 and 20.2, or
(8) 3.3, 8.4, 9.9, 11.8, 12.7, 14.0, 14.8, 16.5, 17.1, 17.9, 19.4, 19.8, 20.2 and 23.5

In one embodiment of the invention, the compound of formula (II) is 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid, which exhibits at least the following characteristic X-ray powder diffraction peaks (expressed in degrees 2θ) (Form A):
(1) 5.1, 10.2 and 12.9, or
(2) 5.1, 8.9 and 13.2, or
(3) 8.9, 10.2, 12.9, 15.1, 17.0 and 21.2 or
(4) 5.1, 8.9, 10.2, 14.6, 15.4, 21.2 and 25.8 or
(5) 5.1, 8.9, 10.2, 12.6, 14.6, 15.1 and 17.0 or
(6) 5.1, 10.2, 12.6, 13.2, 14.6, 15.1, 17.0, 17.9, 21.2 and 21.8 or
(7) 5.1, 8.9, 10.2, 12.6, 13.2, 14.6, 14.9, 16.4, 19.2, 21.8 and 27.1 or
(8) 5.1, 8.9, 10.2, 12.6, 12.9, 13.2, 14.6, 14.9, 15.1, 15.4, 16.4, 17.9, 19.2, 20.0, 21.8 and 25.8.

In another embodiment of the invention, the compound of formula (II) is 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid, which exhibits at least the following characteristic X-ray powder diffraction peaks (expressed in degrees 2θ) (Form C):
(1) 4.5, 8.9 and 12.8, or
(2) 4.5, 8.6 and 10.6, or
(3) 4.5, 8.9, 10.6, 12.8, 14.8 and 17.6 or
(4) 8.6, 8.9, 12.8, 13.9, 15.7, 16.6 and 18.8 or
(5) 4.5, 8.6, 8.9, 10.6, 13.9, 15.7, 16.0, 16.6 and 17.9 or
(6) 4.5, 8.9, 10.6, 12.8, 13.9, 14.8, 15.7, 17.6, 18.8 and 20.0 or
(7) 4.5, 8.6, 8.9, 10.6, 12.8, 13.9, 15.7, 16.0, 16.6, 17.9, 18.8, 20.0, 20.9 and 21.2.

The preparation of these polymorphic salts is described in WO2008/010765.

For the avoidance of doubt, the present invention relates to a pharmaceutical product whereby the muscarinic antagonist is combined with any of the salts or specific polymorphs of compounds of formula (I) or (II) as defined above.

The compounds of formula (I) according to the present invention may be prepared using the processes set out in WO01/98273, WO03/051839 and WO 2005/037814.

The compounds of formula (II) according to the present invention may be prepared using the process set out in WO2004/005295, WO2008/010765 and WO 2004/005295.

The compounds of formula (I) and (II) or a pharmaceutically acceptable or a pharmaceutically acceptable salt, solvates or solvated salt thereof, as defined above may also be prepared according to the preparation routes described in schemes 1 to 4 below.

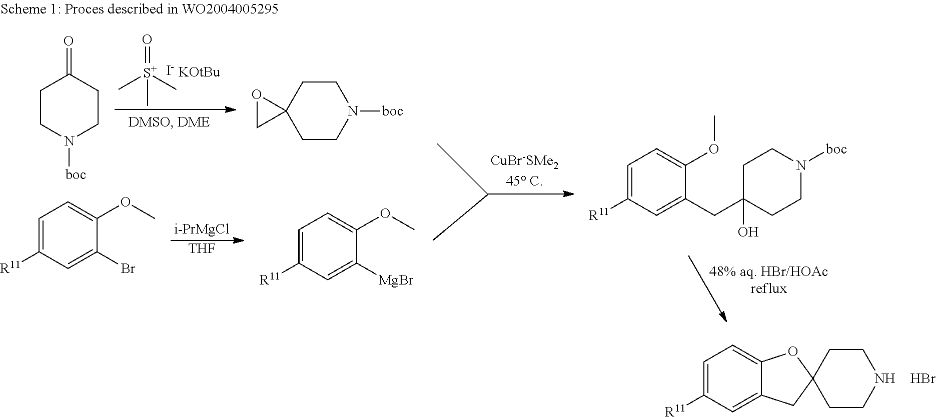

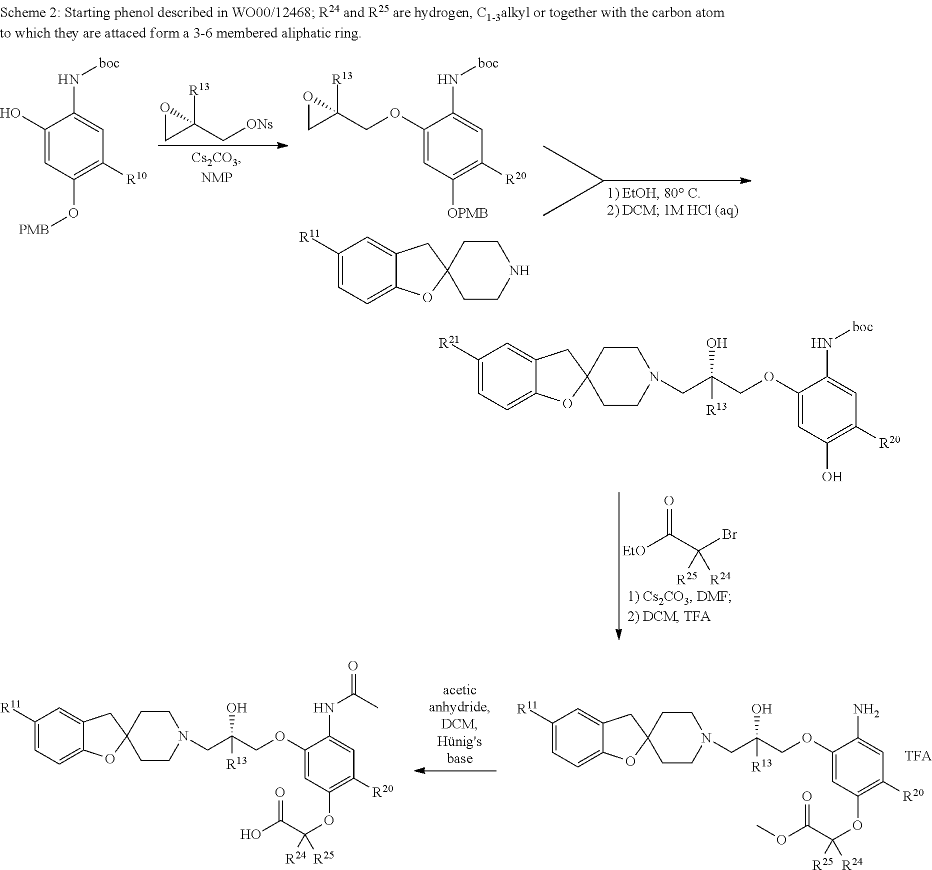

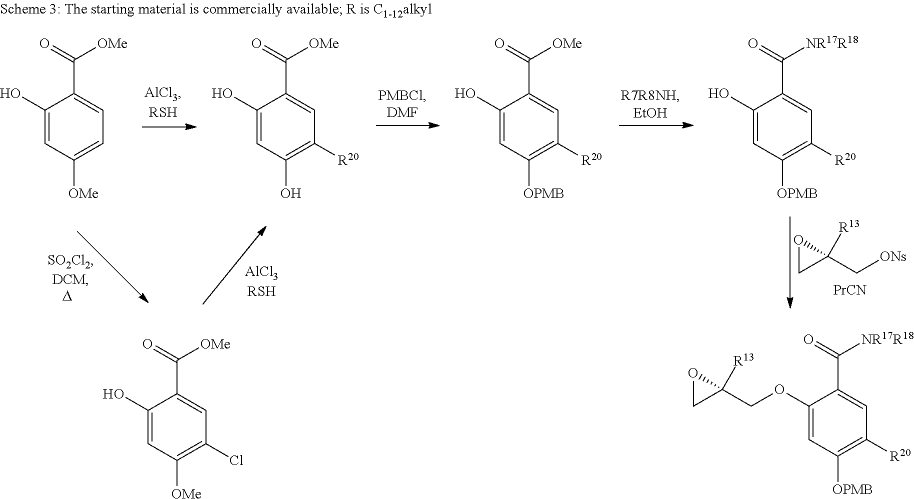

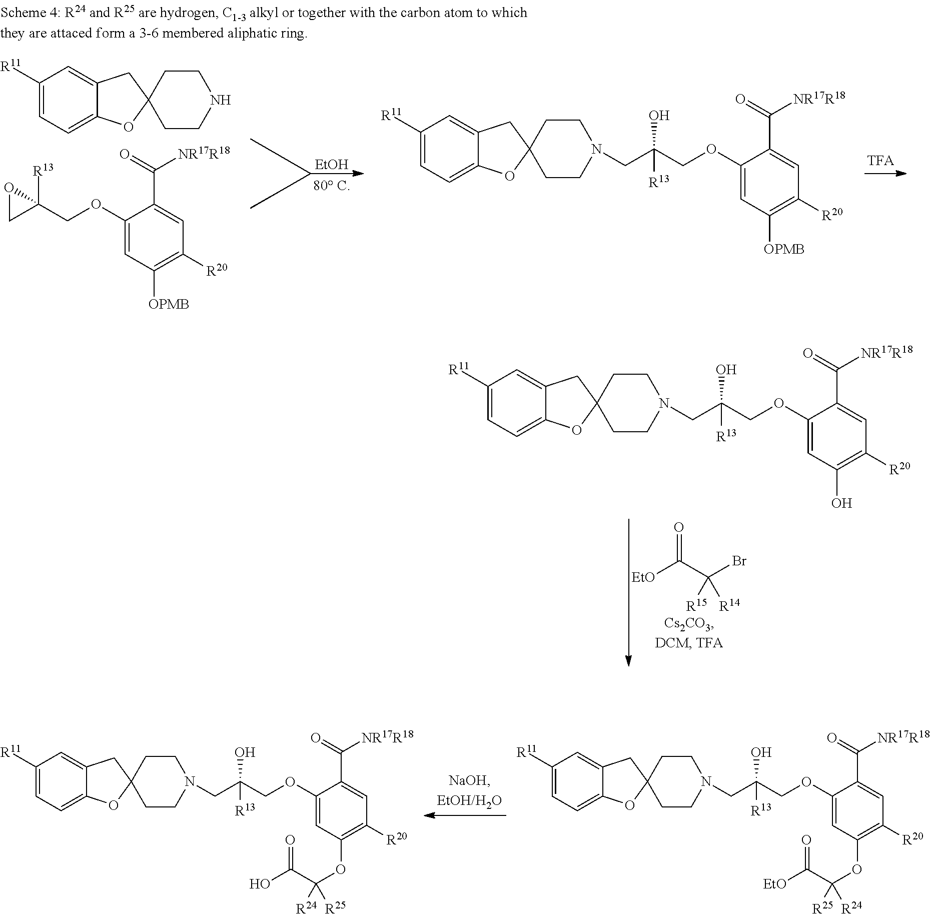

The second active ingredient in the combination of the present invention is a muscarinic antagonist. One embodiment of the invention relates to long acting muscarinic antagonists. Another embodiment relates to short acting muscarinic antagonists.

Non-limiting examples of a muscarinic antagonist that may be used in the pharmaceutical product according to the present invention include ipratropium (e.g. as bromide), tiotropium (e.g. as bromide), oxitropium (e.g. as bromide), tolterodine, pirenzepine, telenzepine, glycopyrronium bromide (such as R,R-glycopyrronium bromide or a mixture of R,S- and S,R-glycopyrronium bromide); mepensolate (e.g. as bromide), a quinuclidine derivative such as 3(R)-(2-hydroxy-2,2-dithien-2-ylacetoxy)-1-(3-phenoxypropyl)-1-azonia-bicyclo[2.2.2]octane bromide as disclosed in US 2003/0055080, quinuclidine derivatives as disclosed in WO 2003/087096 and WO 2005/115467 and DE 10050995; or is GSK 656398 or GSK 961081.

Muscarinic antagonists according to the present invention include ammonium salts as described in WO 2007/017669 and WO2007/017670.

In an embodiment of the invention the muscarinic antagonist is selected from:
[2-(Hydroxy-diphenyl-methyl)-oxazol-5-ylmethyl]-dimethyl-(3-phenoxy-propyl)-ammonium salts,
[2-((R)-Cyclohexyl-hydroxy-phenyl-methyl)-oxazol-5-ylmethyl]-dimethyl-phenethyl-ammonium salts,
[2-((R)-Cyclohexyl-hydroxy-phenyl-methyl)-oxazol-5-ylmethyl]-dimethyl-(4-methyl-pent-3-enyl)-ammonium salts,
[2-((R)-Cyclohexyl-hydroxy-phenyl-methyl)-oxazol-5-ylmethyl]-[2-(2,3-dihydro-benzofuran-5-yl)-ethyl]-dimethyl-ammonium salts,
[2-((R)-cyclohexyl-hydroxy-phenyl-methyl)-oxazol-5-ylmethyl]-dimethyl-(6-methyl-pyridin-2-ylmethyl)-ammonium salts,
[2-(Cyclopentyl-hydroxy-phenyl-methyl)-oxazol-5-ylmethyl]-dimethyl-(3-phenoxy-propyl)-ammonium salts,
[2-(Cyclopentyl-hydroxy-phenyl-methyl)-oxazol-5-ylmethyl]-dimethyl-(3-phenoxy-propyl)-ammonium salts,
1-[2-(Cyclohexyl-hydroxy-phenyl-methyl)-oxazol-5-ylmethyl]-1-(3-phenoxy-propyl)-pyrrolidinium salts,
[2-((R)-Cyclohexyl-hydroxy-phenyl-methyl)-oxazol-5-ylmethyl]-dimethyl-(4-phenoxy-butyl)-ammonium salts,
(2-Benzyloxy-ethyl)-[2-((R)-cyclohexyl-hydroxy-phenyl-methyl)-oxazol-5-ylmethyl]-dimethyl-ammonium salts,
[2-((R)-Cyclohexyl-hydroxy-phenyl-methyl)-oxazol-5-ylmethyl]-dimethyl-(4-phenyl-butyl)-ammonium salts,
[2-((R)-Cyclohexyl-hydroxy-phenyl-methyl)-oxazol-5-ylmethyl]-[3-(4-fluoro-phenoxy)-propyl]-dimethyl-ammonium salts,
[2-((R)-cyclohexyl-hydroxy-phenyl-methyl)-oxazol-5-ylmethyl]-dimethyl-(3-phenyl-propyl)-ammonium salts,
[2-((R)-cyclohexyl-hydroxy-phenyl-methyl)-oxazol-5-ylmethyl]-dimethyl-(2-phenoxy-ethyl)-ammonium salts,
[2-((R)-Cyclohexyl-hydroxy-phenyl-methyl)-oxazol-5-ylmethyl]-dimethyl-(3-p-tolyloxy-propyl)-ammonium salts,
[3-(4-Chloro-phenoxy)-propyl]-[2-((R)-cyclohexyl-hydroxy-phenyl-methyl)-oxazol-5-ylmethyl]-dimethyl-ammonium salts,
[2-((R)-cyclohexyl-hydroxy-phenyl-methyl)-oxazol-5-ylmethyl]-dimethyl-(8-methylamino-octyl)-ammonium salts,
[2-((R)-cyclohexyl-hydroxy-phenyl-methyl)-oxazol-5-ylmethyl]-dimethyl-[2-(4-methylaminomethyl-phenyl)-ethyl]-ammonium salts,
{2-[2-(Cyclohexyl-hydroxy-phenyl-methyl)-oxazol-5-yl]-ethyl}-dimethyl-(3-phenoxy-propyl)-ammonium salts,
{2-[2-(Cyclohexyl-hydroxy-phenyl-methyl)-oxazol-5-yl]-ethyl}-dimethyl-(3-phenoxy-propyl)-ammonium salts,
{2-[2-(Hydroxy-diphenyl-methyl)-oxazol-5-yl]-ethyl}-dimethyl-(3-phenoxy-propyl)-ammonium salts,
[2-(Hydroxydiphenylmethyl)thiazol-5-ylmethyl]dimethyl-(3-phenoxypropyl)ammonium salts,
(3-Benzyloxypropyl)-[2-((R)-cyclohexyl-hydroxy-phenyl-methyl)-oxazol-5-ylmethyl]-dimethyl-ammonium salts,
anti-2-(Biphenyl-2-ylcarbamoyloxy)bicyclo[2.2.1]hept-7-yl]-dimethyl-(3-phenoxy-propyl)-ammonium salts,
anti-[(1S,2R)-2-(2-Hydroxy-2,2-di-thiophen-2-yl-acetoxy)-bicyclo[2.2.1]kept-7-yl]-dimethyl-(3-phenyl-propyl)-ammonium salts,
anti-(±)-2-(2-Hydroxy-2,2-di-thiophen-2-yl-acetoxy)-bicyclo[2.2.1]hept-7-yl]-dimethyl-(3-phenyl-propyl)-ammonium salts,
anti-(1S,2R)-2-(2-Hydroxy-2,2-di-thiophen-2-yl-acetoxy)-bicyclo[2.2.1]hept-7-yl]-dimethyl-(3-phenoxy-propyl)-ammonium salts,
anti-[(1S,2R)-2-(2-Hydroxy-2,2-di-thiophen-2-yl-acetoxy)-bicyclo[2.2.1]hept-7-yl]-dimethyl-phenethyl-ammonium salts,
anti-[(1S,2R)-2-(2-Hydroxy-2,2-di-thiophen-2-yl-acetoxy)-bicyclo[2.2.1]kept-7-yl]-dimethyl-(4-phenyl-butyl)-ammonium salts,
anti-[(1S,2R)-2-(2-Hydroxy-2,2-di-thiophen-2-yl-acetoxy)-bicyclo[2.2.1]hept-7-yl]-trimethyl-ammonium salts,
(2-Benzyloxy-ethyl)-anti-[(1S,2R)-2-(2-hydroxy-2,2-di-thiophen-2-yl-acetoxy)-bicyclo[2.2.1]hept-7-yl]-dimethyl-ammonium salts,
anti-(1S,2R)2-(2-Hydroxy-2,2-diphenyl-acetoxy)-bicyclo[2.2.1]hept-7-yl]-dimethyl-(3-phenoxy-propyl)-ammonium salts,
anti-(1S,2R) Dimethyl-(3-phenoxy-propyl)-[2-(9H-xanthene-9-carbonyloxy)-bicyclo[2.2.1]hept-7-yl]-ammonium salts,
anti-(1S,2R)2-(9-Hydroxy-9H-xanthene-9-carbonyloxy)-bicyclo[2.2.1]hept-7-yl]-dimethyl-(3-phenoxy-propyl)-ammonium salts, and
anti-[(1S,2R)-2-(2-Hydroxy-2,2-di-thiophen-2-yl-acetoxy)-bicyclo[2.2.1]hept-7-yl]-indan-2-yl-dimethyl-ammonium salts. These compounds may be prepared by processes described in patent applications WO 2007/017669 and WO2007/017670.

In another embodiment of the invention the muscarinic antagonist is selected from:
a [2-((S)-Cyclohexyl-hydroxy-phenyl-methyl)-oxazol-5-ylmethyl]-dimethyl-(3-phenoxy-propyl)-ammonium salt,
a [2-((R)-Cyclohexyl-hydroxy-phenyl-methyl)-oxazol-5-ylmethyl]-dimethyl-(3-phenoxy-propyl)-ammonium salt,
a [2-((R)-Cyclohexyl-hydroxy-phenyl-methyl)-oxazol-5-ylmethyl]-dimethyl-(2-phenethyloxy-ethyl)-ammonium salt,
a [2-((R)-Cyclohexyl-hydroxy-phenyl-methyl)-oxazol-5-ylmethyl]-[3-(3,4-dichloro-phenoxy)-propyl]-dimethyl-ammonium salt,
a [2-((R)-Cyclohexyl-hydroxy-phenyl-methyl)-oxazol-5-ylmethyl]-[2-(3,4-dichloro-benzyloxy)-ethyl]-dimethyl-ammonium salt; or
a [2-(4-Chloro-benzyloxy)-ethyl]-[2-((R)-Cyclohexyl-hydroxy-phenyl-methyl)-oxazol-5-ylmethyl]-dimethyl-ammonium salt.

The names of the muscarinc antagonists recited in this embodiment are IUPAC names generated by the Autonom 2000 plug in for IsisDraw Version 2.5, as supplied by MDL Information Systems Inc., with stereochemistry assigned according to the Cahn-Ingold-Prelog system. These compounds may be prepared by processes described in patent application WO 2007/017669.

For the avoidance of doubt, the present invention relates to a pharmaceutical product whereby any one of the compounds falling within the scope of formula (I) or (II) as defined above or any one of the compounds or salts or polymorphs of compounds of formula (I) or (II) mentioned above is combined with any one of the specific the muscarinic antagonist mentioned above.

One embodiment relates to a combination wherein the first active ingredient is a CCR1 antagonist and the second active ingredient is a muscarinic antagonist, with the provision that the muscarinic antagonist is not selected from
a 2-((S)-Cyclohexyl-hydroxy-phenyl-methyl)-oxazol-5-ylmethyl]-dimethyl-(3-phenoxy-propyl)-ammonium salt,
a [2-((R)-Cyclohexyl-hydroxy-phenyl-methyl)-oxazol-5-ylmethyl]-dimethyl-(3-phenoxy-propyl)-ammonium salt,
a [2-((R)-Cyclohexyl-hydroxy-phenyl-methyl)-oxazol-5-ylmethyl]-dimethyl-(2-phenethyloxy-ethyl)-ammonium salt,
a [2-((R)-Cyclohexyl-hydroxy-phenyl-methyl)-oxazol-5-ylmethyl]-[3-(3,4-dichloro-phenoxy)-propyl]-dimethyl-ammonium salt,
a [2-((R)-Cyclohexyl-hydroxy-phenyl-methyl)-oxazol-5-ylmethyl]-[2-(3,4-dichloro-benzyloxy)-ethyl]-dimethyl-ammonium salt, or
a [2-(4-Chloro-benzyloxy)-ethyl]-[2-((R)-Cyclohexyl-hydroxy-phenyl-methyl)-oxazol-5-ylmethyl]-dimethyl-ammonium salt.

In one embodiment, the muscarinic antagonist according to the present invention include, aclidinium bromide, glycopyrrolate (such as R,R-, R,S-, S,R-, or S,S-glycopyrronium bromide), oxitropium bromide, pirenzepine, telenzepine, 3(R)-(2-hydroxy-2,2-dithien-2-ylacetoxy)-1-(3-phenoxypropyl)-1-azoniabicyclo[2.2.2]octane bromide, 3(R)-1-phenethyl-3-(9H-xanthene-9-carbonyloxy)-1-azoniabicyclo[2.2.2]octane bromide and (3R)-3-[(2S)-2-cyclopentyl-2-hydroxy-2-thien-2-ylacetoxy]-1-(2-phenoxyethyl)-1-azoniabicyclo[2.2.2]octane bromide.

In the context of the present specification, unless otherwise indicated any reference to a muscarinic antagonist includes all active salts, solvates or derivatives that may be formed from said muscarinic antagonist. Examples of possible salts or derivatives of muscarinic antagonist include; sodium salts, bromides, sulphobenzoates, phosphates, isonicotinates, acetates, propionates, dihydrogen phosphates, palmitates, pivalates, fumarates and pharmaceutically acceptable esters (e.g. C₁₋₆alkyl esters). Muscarinic antagonist and active salts or derivatives thereof may also be in the form of their solvates, e.g. hydrates as well as cocrystals.

The anion of the ammonium salt may be any pharmaceutically acceptable anion of a mono or polyvalent (e.g. bivalent) acid, examples include chloride, bromide, iodide, sulfate, benzenesulfonate, toluenesulfonate (tosylate), napadisylate (naphthalene-1,5-disulfonate e.g. hemi-napadiylate), edisylate (ethane-1,2-disulfonate), isethionate (2-hydroxyethylsulfonate), phosphate, acetate, citrate, lactate, tartrate, oleic, mesylate (methanesulfonate), maleate ((Z)-3-carboxy-acrylate), fumarate, succinate (3-carboxy-propionate), malate ((S)-3-carboxy-2-hydroxy-propionate), xinafoate and p-acetamidobenzoate.

### Pharmaceutical Compositions

The active ingredients of the present invention may be administered by oral or parenteral (e.g. intravenous, subcutaneous, intramuscular or intraarticular) administration using conventional systemic dosage forms, such as tablets, capsules, pills, powders, aqueous or oily solutions or suspensions, emulsions and sterile injectable aqueous or oily solutions or suspensions. The active ingredients may also be administered topically (e.g. to the lung and/or airways) in the form of solutions, suspensions, aerosols and dry powder compositions. These dosage forms will usually include one or more pharmaceutically acceptable ingredients which may be selected, for example, from adjuvants, carriers, binders, lubricants, diluents, stabilising agents, buffering agents, emulsifying agents, viscosity-regulating agents, surfactants, preservatives, flavourings and colorants. As will be understood by those skilled in the art, the most appropriate method of administering the active ingredients is dependent on a number of factors.

One embodiment relates to a pharmaceutical composition comprising, in admixture, a first active ingredient which is a compound of formula (I) or (II) (i.e. any one of the compounds falling within the scope of formula (I) or (II) as defined above or any one of the compounds or salts or polymorphs of compounds of formula (I) or (II) mentioned above) or a pharmaceutically acceptable salt thereof, and a second active ingredient which is a muscarinic antagonist mentioned above, in admixture with pharmaceutically acceptable adjuvants, diluents and/or carriers.

In one embodiment of the present invention the active ingredients are administered via separate pharmaceutical compositions.

Therefore, in one aspect, the present invention provides a kit comprising a composition of a first active ingredient, which is a compound of formula (I) or (II) (i.e. any one of the compounds falling within the scope of formula (I) or (II) as defined above or any one of the compounds or salts or polymorphs of compounds of formula (I) or (II) mentioned above) or a pharmaceutically acceptable salt thereof and a composition of a second active ingredient, which is a muscarinic antagonist mentioned above, and optionally instructions for the simultaneous, sequential or separate administration of the compositions to a patient in need thereof.

The pharmaceutical compositions of the present invention may be prepared by mixing the first active ingredient and the second active ingredient with a pharmaceutically acceptable adjuvant, diluent or carrier. Therefore, in a further aspect of the present invention there is provided a process for the preparation of a pharmaceutical composition, which comprises mixing a compound of formula (I) or (II), ad defined above, or pharmaceutically acceptable salt thereof, with a second active ingredient as defined above, and a pharmaceutically acceptable adjuvant, diluent or carrier.

It will be understood that the therapeutic dose of each active ingredient administered in accordance with the present invention will vary depending upon the particular active ingredient employed, the mode by which the active ingredient is to be administered, and the condition or disorder to be treated.

In one embodiment of the present invention, the first and second active ingredients of the present invention are each administered by inhalation. In this embodiment, the active ingredients may be inhaled simultaneously. In another embodiment the active ingredients may be inhaled sequentially. Or in a further embodiment the active ingredients may be inhaled separately.

The active ingredients are conveniently administered via inhalation (e.g. topically to the lung and/or airways) in the form of solutions, suspensions, aerosols or dry powder compositions. Administration may be by inhalation, orally or intranasally. The active ingredients are preferably adapted to be administered, either together or individually, from a dry powder inhaler, pressurised metered dose inhaler, or a nebuliser.

The active ingredients may be used in admixture with one or more pharmaceutically acceptable additives, diluents or carriers. Examples of suitable diluents or carriers include lactose (e.g. the monohydrate), dextran, mannitol or glucose.

Metered dose inhaler devices may be used to administer the active ingredients, dispersed in a suitable propellant and with or without additional excipients such as ethanol, a surfactant, a lubricant, an anti-oxidant or a stabilising agent. Suitable propellants include hydrocarbon, chlorofluorocarbon and hydrofluoroalkane (e.g. heptafluoroalkane) propellants, or is mixtures of any such propellants. Preferred propellants are P134a and P227, each of which may be used alone or in combination with other propellants and/or surfactant and/or other excipients. Nebulised aqueous suspensions, solutions may also be employed, with or without a suitable pH and/or tonicity adjustment, either as a unit-dose or multi-dose compositions.

Dry powder inhalers may be used to administer the active ingredients, alone or in combination with a pharmaceutically acceptable carrier, in the later case either as a finely divided powder or as an ordered mixture. The dry powder inhaler may be single dose or multi-dose and may utilise a dry powder or a powder-containing capsule.

When the active ingredients are adapted to be administered, either together or individually, via a nebuliser they may be in the form of a nebulised aqueous suspension or solution, with or without a suitable pH or tonicity adjustment, either as a single dose or multidose device.

Metered dose inhaler, nebuliser and dry powder inhaler devices are well known and a variety of such devices are available.

In one embodiment, the present invention provides a pharmaceutical product comprising, in combination, a first active ingredient which is a compound of formula (I) or (II), (i.e. any one of the compounds falling within the scope of formula (I) or (II) as defined above or any one of the compounds or salts or polymorphs of compounds of formula (I) or (II) mentioned above) or a pharmaceutically acceptable salt thereof, and a second active ingredient, which is muscarinic antagonist, wherein each active ingredient is formulated for inhaled administration.

In another embodiment of the present invention, the first active ingredient, which is a compound of formula (I) or (II), as defined above, or a pharmaceutically acceptable salt thereof, may be formulated for oral administration and the second active ingredient(s), which is a muscarinic antagonist, as defined above, may be formulated for inhaled administration.

In yet another embodiment of the present invention, the first active ingredient, which is a compound of formula (I) or (II), as defined above, or a pharmaceutically acceptable salt thereof, may be formulated for inhaled administration and the second active ingredient(s), which is a muscarinic antagonist, as defined above, may be formulated for oral administration.

In yet a further embodiment of the present invention, the first active ingredient, which is a compound of formula (I) or (II), as defined above, or a pharmaceutically acceptable salt thereof, and the second active ingredient(s), which is a muscarinic antagonist, as defined above, wherein each active ingredient is formulated for oral administration.

### Medical Use

The use of compounds of formula (I) or (II) are contemplated to demonstrate particular effects when used in combination with a muscarinic antagonist, and in particular in combination with tiotropium. For example, in vivo animal experiments indicate that a combination of a muscarinic antagonist and a compound of formula (I) or (II), at dose levels where neither component alone significantly affects lung inflammation, in combination give significant reduction of inflammatory cell influx. The reduction in cell influx for the combination is greater than that expected from the additive effect of the two ingredients. This synergistic effect observed when combining the ingredients could be used, for example, to lower the therapeutic dose of muscarinic antagonist, or at the same dose, achieve enhanced efficacy on inflammation in comparison to the use of the muscarinic antagonist alone. The synergistic effect can be particularly advantageous where lower doses of the muscarinic antagonist are desirable, for example in individuals that have acquired resistance to such a muscarinic antagonist.

Examples of conditions diseases which may be treated using the combination of the invention are, but not limited to, airways/respiratory diseases including chronic obstructive pulmonary disease (COPD) such as irreversible COPD; asthma, such as bronchial, allergic, intrinsic, extrinsic and dust asthma, particularly chronic or inveterate asthma (e.g. late asthma and airways hyper-responsiveness); bronchitis; acute, allergic, atrophic rhinitis and chronic rhinitis including rhinitis caseosa, hypertrophic rhinitis, rhinitis purulenta, rhinitis sicca and rhinitis medicamentosa; membranous rhinitis including croupous, fibrinous and pseudomembranous rhinitis and scrofoulous rhinitis; seasonal rhinitis including rhinitis nervosa (hay fever) and vasomotor rhinitis; sarcoidosis, farmer's lung and related diseases, fibroid lung and idiopathic interstitial pneumonia.

The compound of formula (I) or (II), or a pharmaceutically acceptable salt thereof, (first active ingredient) and the muscarinic antagonist or a pharmaceutically acceptable salt thereof, (second active ingredient) may be administered simultaneously, sequentially or separately to treat airway diseases. By sequential it is meant that the active ingredients are administered, in any order, one immediately after the other. They still have the desired effect if they are administered separately, but when administered in this manner they are generally administered less than 4 hours apart, more conveniently less than two hours apart, more conveniently less than 30 minutes apart and most conveniently less than 10 minutes apart.

Throughout the specification, the amount of the active ingredients used relate to unit doses unless explicitly defined differently.

When administered via inhalation the dose of the first active ingredient (compound of formula (I) or (II) or a pharmaceutically acceptable salt thereof), will generally be in the range of from 0.1 μg to 10000 μg, 0.1 to 5000 μg, 0.1 to 1000 μg, 0.1 to 500 μg, 0.1 to 200 μg, 0.1 to 200 μg, 0.1 to 100 μg, 0.1 to 50 μg, 5 μg to 5000 μg, 5 to 1000 μg, 5 to 500 μg, 5 to 200 μg, 5 to 100 μg, 5 to 50 μg, 10 to 5000 μg, 10 to 1000 μg, 10 to 500 μg, 10 to 200 μg, 10 to 100 μg, 10 to 50 μg, 20 to 5000 μg, 20 to 1000 μg, 20 to 500 μg, 20 to 200 μg, 20 to 100 μg, 20 to 50 μg, 50 to 5000 μg, 50 to 1000 μg, 50 to 500 μg, 50 to 200 μg, 50 to 100 μg, 100 to 5000 μg, 100 to 1000 μg or 100 to 500 μg.

In one embodiment, the amount of the first active ctive ingredient used is in the range of from 1 μg to 200 μg, and that of the second active ingredient is in the range of from 1 μg to 200 μg.

When administered via inhalation the dose of the second active ingredient (muscarinic antagonist), will generally be in the range of from 0.1 microgram (μg) to 1000 μg, 0.1 to 500 μg, 0.1 to 200 μg, 0.1 to 100 μg, 0.1 to 50 μg, 0.1 to 5 μg, 5 to 1000 μg, 5 to 500 μg, 5 is to 200 μg, 5 to 50 μg, 5 to 10 μg, 10 to 1000 μg, 10 to 500 μg, 10 to 200 μg, 10 to 100 μg, 10 to 50 μg, 20 to 1000 μg, 20 to 500 μg, 20 to 200 μg, 20 to 100 μg, 20 to 50 μg, 50 to 1000 μg, 50 to 500 μg, 50 to 200 μg, 50 to 100 μg, 100 to 1000 μg, or 100 to 500 μg.

The molar ratio of the second active ingredient to the first active ingredient in a dose may typically be in the range of from 300:1 to 1:300. In one embodiment the ratio is in the range of from 100:1 to 1:100. In another embodiment the ratio is in the range of from 50:1 to 1:50. In a further embodiment the ratio is in the range of from 10:1 to 1:10. In yet another embodiment the ratio is in the range of from 5:1 to 1:5.

In one embodiment the ratio is in the range of 1:10 to 1:50. In another embodiment the ratio is in the range of 1:15 to 1:40.

The M3 antagonists are likely to have a lower molecular weight but may be as potent at their receptor as the CCR1 antagonists.

The doses of the first and second active ingredients will generally be administered from 1 to 4 times a day, conveniently once or twice a day, and most conveniently once a day.

The present invention further provides a pharmaceutical product, kit or pharmaceutical composition comprising the combination according to the present invention for simultaneous, sequential or separate use in therapy.

The present invention further provides the use of a pharmaceutical product, kit or pharmaceutical composition, which comprises:
(a) a (therapeutically effective) dose of a first active ingredient, which is a compound of formula (I) or (II), (i.e. any one of the compounds falling within the scope of formula (I) or (II) as defined above or any one of the compounds or salts or polymorphs of compounds of formula (I) or (II) mentioned above) or a pharmaceutically acceptable salt thereof; and
(b) a (therapeutically effective) dose of a second active ingredient, which is a muscarinic antagonist, or a pharmaceutically acceptable salt thereof, provided that the muscarinic antagonist is not selected from
a 2-((S)-Cyclohexyl-hydroxy-phenyl-methyl)-oxazol-5-ylmethyl]-dimethyl-(3-phenoxy-propyl)-ammonium salt,
a [2-((R)-Cyclohexyl-hydroxy-phenyl-methyl)-oxazol-5-ylmethyl]-dimethyl-(3-phenoxy-propyl)-ammonium salt,
a [2-((R)-Cyclohexyl-hydroxy-phenyl-methyl)-oxazol-5-ylmethyl]-dimethyl-(2-phenethyloxy-ethyl)-ammonium salt,
a [2-((R)-Cyclohexyl-hydroxy-phenyl-methyl)-oxazol-5-ylmethyl]-[3-(3,4-dichloro-phenoxy)-propyl]-dimethyl-ammonium salt,
a [2-((R)-Cyclohexyl-hydroxy-phenyl-methyl)-oxazol-5-ylmethyl]-[2-(3,4-dichloro-benzyloxy)-ethyl]-dimethyl-ammonium salt, or
a [2-(4-Chloro-benzyloxy)-ethyl]-[2-((R)-Cyclohexyl-hydroxy-phenyl-methyl)-oxazol-5-ylmethyl]-dimethyl-ammonium salt, in the manufacture of a medicament for the treatment of airway diseases.

The present invention further provides the use of a pharmaceutical product, kit or pharmaceutical composition, which comprises:
(a) a (therapeutically effective) dose of a first active ingredient, which is a compound of formula (I) or (II), (i.e. any one of the compounds falling within the scope of formula (I) or (II) as defined above or any one of the compounds or salts or polymorphs of compounds of formula (I) or (II) mentioned above) or a pharmaceutically acceptable salt thereof; and
(b) a (therapeutically effective) dose of a second active ingredient, which is a muscarinic antagonist, or a pharmaceutically acceptable salt thereof as defined above, in the manufacture of a medicament for the treatment of chronic obstructive pulmonary disease or asthma, or any other disorder mentioned above.

The present invention still further provides a method of treating airway diseases, or chronic obstructive pulmonary disease or asthma, or any other disorder mentioned above which comprises simultaneously, sequentially or separately administering:
(a) a (therapeutically effective) dose of a first active ingredient, which is a compound of formula (I) or (II), (i.e. any one of the compounds falling within the scope of formula (I) or (II) as defined above or any one of the compounds or salts or polymorphs of compounds of formula (I) or (II) mentioned above) or a pharmaceutically acceptable salt thereof; and
(b) a (therapeutically effective) dose of a second active ingredient, which is a muscarinic antagonist, or a pharmaceutically acceptable salt thereof as defined above,
to a patient in need thereof, provided that the muscarinic antagonist is not selected from
a 2-((S)-Cyclohexyl-hydroxy-phenyl-methyl)-oxazol-5-ylmethyl]-dimethyl-(3-phenoxy-propyl)-ammonium salt,
a [2-((R)-Cyclohexyl-hydroxy-phenyl-methyl)-oxazol-5-ylmethyl]-dimethyl-(3-phenoxy-propyl)-ammonium salt,
a [2-((R)-Cyclohexyl-hydroxy-phenyl-methyl)-oxazol-5-ylmethyl]-dimethyl-(2-phenethyloxy-ethyl)-ammonium salt,
a [2-((R)-Cyclohexyl-hydroxy-phenyl-methyl)-oxazol-5-ylmethyl]-[3-(3,4-dichloro-phenoxy)-propyl]-dimethyl-ammonium salt,
a [2-((R)-Cyclohexyl-hydroxy-phenyl-methyl)-oxazol-5-ylmethyl]-[2-(3,4-dichloro-benzyloxy)-ethyl]-dimethyl-ammonium salt, or
a [2-(4-Chloro-benzyloxy)-ethyl]-[2-((R)-Cyclohexyl-hydroxy-phenyl-methyl)-oxazol-5-ylmethyl]-dimethyl-ammonium salt.

One embodiment relates to the uses and methods described above wherein the first active ingredient is any one of N-{2-[((2S)-3-{[1-(4-chlorobenzyl)piperidin-4-yl]amino}-2-hydroxy-2-methylpropyl)oxy]-4-hydroxyphenyl}acetamide or N-{5-Chloro-2-[((2S)-3-{[1-(4-chlorobenzyl)piperidin-4-yl]amino}-2-hydroxy-2-methylpropyl)oxy]-4-hydroxyphenyl}acetamide or 2-{2-Chloro-5-{[(2S)-3-(5-chloro-1′H,3H-spiro[1-benzofuran-2,4′-piperidin]-1′-yl)-2-hydroxypropyl]oxy}-4[(methylamino)carbonyl]phenoxy}-2-methylpropanoic acid or a pharmaceutically acceptable salt thereof, and
(b) a second active ingredient is tiotropium, or a pharmaceutically acceptable salt thereof.

In another embodiment second active ingredient is tiotropium bromide.

One embodiment of the invention relates to the combination as described above wherein phosphodiesterase (PDE) inhibitors as well as glucocorticoid receptor agonists are excluded from the combination of the invention.

In the context of the present specification, the term “therapy” also includes “prophylaxis” unless there are specific indications to the contrary. The terms “therapeutic” and “therapeutically” should be construed accordingly. Prophylaxis is expected to be particularly relevant to the treatment of persons who have suffered a previous episode of, or are otherwise considered to be at increased risk of, the condition or disorder in question. Persons at risk of developing a particular condition or disorder generally include those having a family history of the condition or disorder, or those who have been identified by genetic testing or screening to be particularly susceptible to developing the condition or disorder.

The term “disease”, unless stated otherwise, has the same meaning as the terms “condition” and “disorder” and are used interchangeably throughout the description and claims.

The term “agent” and “ingredient” means the compounds comprised in the combination of the present invention, i.e. a CCR1 antagonist or a muscarinic antagonist.

### EXAMPLES

The present invention will now be further understood by reference to the following illustrative examples.

The following abbreviations are used:

### APCI-MS Atmospheric Pressure Chemical Ionisation Mass Spectroscopy;

### DCM Dichloromethane

### DIEA N,N-Diisopropylethylamine;

### DMF N,N-Dimethylformamide

### DMSO Dimethylsulfoxide;

### HPLC High Performance Liquid Chromatography;

### LC/MS Liquid Column Chromatography/Mass Spectroscopy;

TFA Trifluoroacetic acid;

### THF Tetrahydrofuran

Eq equivalent
EtOH ethanol

### EtOAc Ethylacetate

### General Methods

¹H NMR and ¹³C NMR spectra were recorded on a Varian Inova 400 MHz or a Varian Mercury-VX 300 MHz instrument. The central peaks of chloroform-d (δ_{H} 7.27 ppm), dimethylsulfoxide-d₆ (δ_{H} 2.50 ppm), acetonitrile-d₃ (δ_{H} 1.95 ppm) or methanol-d₄ (δ_{H} 3.31 ppm) were used as internal references. Flash chromatography was carried out using silica gel (0.040-0.063 mm, Merck). Unless stated otherwise, starting materials were commercially available. All solvents and commercial reagents were of laboratory grade and were used as received.

The following method was used for LC/MS analysis:

Instrument Agilent 1100; Column Waters Symmetry 2.1×30 mm; Mass APCI; Flow rate 0.7 ml/min; Wavelength 254 nm; Solvent A: water+0.1% TFA; Solvent B: acetonitrile+0.1% TFA; Gradient 15-95%/B 2.7 min, 95% B 0.3 min.

The following method was used for LC analysis:

Method A. Instrument Agilent 1100; Column: Kromasil C18 100×3 mm, 5μ particle size,
Solvent A: 0.1% TFA/water, Solvent B: 0.08% TFA/acetonitrile Flow: 1 ml/min, Gradient 10-100% B 20 min, 100% B 1 min. Absorption was measured at 220, 254 and 280 nm.
Method B. Instrument Agilent 1100; Column: XTerra C8, 100×3 mm, 5 μA particle size,
Solvent A: 15 mM NH₃/water, Solvent B: acetonitrile Flow: 1 ml/min, Gradient 10-100% B 20 min, 100% B 1 min. Absorption was measured at 220, 254 and 280 nm.
X-ray powder diffraction (XRPD) analyses may be performed on samples prepared according to standard methods (see for example Giacovazzo et al., eds., Fundamentals of Crystallography, Oxford University Press (1992); Jenkins & Snyder, eds., Introduction to X-Ray Powder Diffractometry, John Wiley & Sons, New York (1996); Bunn, ed., Chemical Crystallography, Clarendon Press, London (1948); and Klug & Alexander eds., X-ray Diffraction Procedures, John Wiley & Sons, New York (1974)).

X-ray powder diffraction patterns of the polymorphic Forms described in Examples 1 and 2 above (in anhydrous form) were obtained as described below:

A Bragg-Brentano parafocusing powder X-ray diffractometer using monochromatic CuKα radiation (45 kV and 40 mA) was used for the analyses. The primary optics contained soller slits and an automatic divergence slit. Flat samples were prepared on zero background plates that were rotated during the measurements. The secondary optics contained soller slits, an automatic anti scatter slit, a receiving slit and a monochromator. The diffracted signal was detected with a proportional xenon-filled detector. Diffraction patterns were collected between 2°≦2θ (theta)≦40° in a continuos scan mode with a step size of 0.016° 2θ at a rate of 4° 2θ per minute. Raw data were stored electronically. Evaluation was performed on raw or smoothed diffraction patterns.

A Panalytical X′ pert PRO MPD θ-θ diffractometer in reflection mode was used for the above-mentioned measurements. A person skilled in the art can set up instrumental parameters for a powder X-ray diffractometer so that diffraction data comparable to the data presented can be collected.

### Example 1

### N-{5-chloro-2-[((2S)-3-{[1-(4-chlorobenzyl)piperidin-4-yl]amino}-2-hydroxy-2-methylpropyl)oxy]-4-hydroxyphenyl}acetamide hemifumarate salt

A 40° C. warm solution of N{5-chloro-2-[((2S)-3-{[1-(4-chlorobenzyl)piperidin-4-yl]amino}-2-hydroxy-2-methylpropyl)oxy]-4-hydroxyphenyl}acetamide (2.62 g) in methanol (15 ml) is added to a 40° C. warm solution of fumaric acid (675 mg) in methanol (10 ml). The solution was allowed to cool to room temperature and the precipitate was collected after 72 h, washed with cold methanol and vacuum-dried, to provide 1.33 g of the titled compound.

¹H NMR (300 MHz, DMSO-d₆) δ 7.62 (m, 2H), 7.47 (m, 2H), 7.27 (s, 1H), 7.11 (s, 1H), 6.98 (s), 6.71 (s), 4.44 (s, 2H), 4.11-4.05 (m, 2H), 3.71-3.55 (m, 4H), 3.39-2.41 (m, 7H), 2.07 (m, 3H), 1.35 (s, 3H); APCI-MS: m/z 496 [MH⁺]; X-ray powder diffraction peaks (expressed in degrees 2θ):
(1) 6.2, 14.7 and 20.5, or
(2) 8.0, 10.1 and 14.7, or
(3) 10.1, 12.4, 14.7 and 19.5, or
(4) 6.2, 10.1, 12.4, 13.4, 19.5 and 20.1, or
(5) 8.0, 10.1, 12.4, 14.7, 19.5, 20.1, 21.2 and 23.8, or
(6) 6.2, 8.0, 10.1, 11.5, 12.4, 13.4, 19.5, 20.1 and 21.2, or
(7) 6.2, 8.0, 11.5, 12.4, 13.4, 14.7, 20.1, 20.5, 21.2 and 23.8, or
(8) 6.2, 8.0, 10.1, 11.5, 12.4, 13.4, 14.7, 16.1, 20.5, 21.2 and 23.8

### Example 2

### N-{5-chloro-2-[((2S)-3-{[1-(4-chlorobenzyl)piperidin-4-yl]amino}-2-hydroxy-2-methylpropyl)oxy]-4-hydroxyphenyl}acetamide sulphate salt

N-{5-chloro-2-[((2S)-3-{[1-(4-chlorobenzyl)piperidin-4-yl]amino}-2-hydroxy-2-methylpropyl)oxy]-4-hydroxyphenyl}acetamide (55 mg) was dissolved in 2-butanol (4 ml) and, under stirring, heated to 55° C. To this a 1M H₂SO₄ in 2-butanol solution (0.11 ml), that is kept at room temperature, was added in one portion. The mixture was warmed to 70° C., additional 2-butanol was added (16 ml) and the suspension stirred for 12 h. The precipitate was filtered off, dried and redissolved in methanol (4 ml). The solution was stirred at room temperature and the solvent allowed to evaporate slowly to the open air, providing the sulphate salt of N-{5-chloro-2-[((2S)-3-{[1-(4-chlorobenzyl)piperidin-4-yl]amino}-2-hydroxy-2-methylpropyl)oxy]-4-hydroxyphenyl}acetamide.

¹H NMR (300 MHz, DMSO-d₆) δ 10.08 (broad), 9.08 (s, 1H), 7.78 (s, 1H), 7.49-7.38 (m, 4H), 6.69 (s, 1H), 3.91 (m, 2H), 3.56 (s, 2H), 3.23-2.81 (m, 5H), 2.13 (s, 3H), 2.09-2.00 (m, 4H), 1.69-1.63 (m, 2H), 1.38 (s, 3H); APCI-MS: m/z 496 [MH⁺]; X-ray powder diffraction peaks (expressed in degrees 2θ):
(1) 3.3, 9.9 and 19.8, or
(2) 8.4, 16.5 and 19.8, or
(3) 3.3, 9.9, 16.5, 17.9, 20.2 and 23.5, or
(4) 9.9, 16.5, 17.1, 17.9, 19.8, 20.2 and 23.5, or
(5) 3.3, 8.4, 9.9, 14.8, 17.1, 19.4, 19.8 and 20.2, or
(6) 9.0, 9.9, 11.8, 14.0, 14.8, 16.5, 19.8, 20.2 and 23.5, or
(7) 3.3, 8.4, 9.0, 9.9, 11.8, 12.7, 14.0, 17.1, 17.9, 19.4, 19.8 and 20.2, or
(8) 3.3, 8.4, 9.9, 11.8, 12.7, 14.0, 14.8, 16.5, 17.1, 17.9, 19.4, 19.8, 20.2 and 23.5

### Example 3

### Methyl 3-(2-{[(2S)-3-{[1-(4-chlorobenzyl)piperidin-4-yl]amino}-2-hydroxypropyl]oxy}-4-fluorophenyl)propanoate

### Step I

### Methyl 3-{4-fluoro-2-[(2S)-oxiran-2-ylmethoxy]phenyl}propanoate

A mixture of (2S)-oxiran-2-ylmethyl 3-nitrobenzenesulfonate (130 mg), methyl 3-(4-fluoro-2-hydroxyphenyl)propanoate (99 mg) and Cs₂CO₃ (196 mg) in DMF (3 ml) was stirred at room temperature for 18 h. The reaction mixture was partitioned between ethyl acetate and water. The organic layer was dried over Na₂SO₄, filtered and concentrated in vacuo. The residue was purified by flash chromatography (0.20% ethyl acetate in petroleum ether 40-60° C.) to give the subtitled compound (105 mg).

¹H-NMR (CDCl₃, 400 MHz): δ 7.10 (t, J=7.5 Hz, 1H); 6.64-6.55 (m, 2H); 4.26 (dd, J=2.8, 11.1 Hz, 1H); 3.93 (dd, J=5.6, 11.1 Hz, 1H); 3.69 (s, 3H); 3.39 (m, 1H); 2.96-2.90 (m, 3H); 2.78 (dd, J=2.7, 4.9 Hz, 1H); 2.60 (t, J=7.7 Hz, 2H).

### Step II

Methyl 3-{4-fluoro-2-[(2S)-oxiran-2-ylmethoxy]phenyl}propanoate (100 mg) and 1-(4-chlorobenzyl)piperidin-4-amine (88 mg) were taken into methanol (3 ml) and the solution stirred at 80° C. for 18 h. The volatiles were removed in vacuo and the residue was purified by flash chromatography (0-2.5% methanol in dichloromethane containing 0.2% NH₄OH) to give the titled compound (100 mg).

¹H-NMR (DMSO-d₆, 400 MHz): δ 7.35 (m, 2H); 7.29 (m, 2H); 7.13 (t, J=7.6 Hz, 1H); 6.84 (dd, J=2.3, 11.4 Hz, 1H); 6.68-6.60 (m, 1H); 4.99 (br.s, 1H); 3.99-3.80 (m, 3H); 3.57 (s, 3H); 3.41 (s, 2H); 2.77 (t, J=7.7 Hz, 2H); 2.74-2.51 (m, 6H); 2.38 (m, 1H); 1.95 (t, J=10.5 Hz, 2H); 1.73 (m, 2H); 1.20 (m, 2H).

APCI-MS: m/z 479 (MH⁺).

### Example 4

### (2-{[(2S)-3-{[1-(4-chlorobenzyl)piperidin-4-yl]amino}-2-hydroxy-2-methylpropyl]oxy}-4-fluorophenyl)methanesulfonic acid

### Step I

### (4-Fluoro-2-{[(2S)-2-methyloxiran-2-yl]methoxy}phenyl)methanol

A mixture of 5-fluoro-2-(hydroxymethyl)phenol (284 mg), [(2S)-2-methyloxiran-2-yl]methyl 3-nitrobenzenesulfonate (546 mg), and Cs₂CO₃ (986 mg) in DMF (5 ml) was stirred at room temperature for 18 h. The mixture was diluted with ethyl acetate (100 ml), and washed with water (2×50 ml). The organic layer was dried with sodium sulphate. The solvent was removed in vacuo to afford subtitle compound, 452 mg, which was used in the next step without further purification.

¹H-NMR (CDCl₃, 400 MHz): δ 7.26 (s, 1H), 6.67 (td, J=8.3, 2.5 Hz, 1H), 6.61 (dd, J=10.4, 2.3 Hz, 1H), 4.67 (dd, J=33.3, 12.7 Hz, 2H), 4.11 (d, J=10.4 Hz, 1H), 4.00 (d, J=10.4 Hz, 1H), 2.94 (d, J=4.6 Hz, 1H), 2.77 (d, J=4.6 Hz, 1H), 1.50 (s, 3H),

### Step II

### tert-Butyl [1-(4-chlorobenzyl)piperidin-4-yl]{(2S)-3-[5-fluoro-2-(hydroxymethyl)phenoxy]-2-hydroxy-2-methylpropyl}carbamate

A mixture of 1-(4-chlorobenzyl)piperidin-4-amine (449 mg) and (4-fluoro-2-{[(2S)-2-methyloxiran-2-yl]methoxy}phenyl)methanol (424 mg) in dry ethanol (15 ml) was heated at 80° C. for 7 h. Then the solution was allowed to cool to room temperature, and di-tert-butyl carbonate (436 mg) was added. The mixture was stirred at room temperature for 18 h, after which the solvent was removed in vacuo. The residue was redissolved in ethyl acetate (50 ml), and washed with water (3×30 ml). The organic layer was dried with sodium sulphate, and after filtration removed in vacuo to afford the subtitle compound as yellowish oil, 1.02 g (95%).

APCI-MS: m/z 537 (MH⁺).

### Step III

### tert-Butyl [1-(4-chlorobenzyl)piperidin-4-yl]{(2S)-3-[2-(chloromethyl)-5-fluorophenoxy]-2-hydroxy-2-methylpropyl}carbamate

Polymer-bound triphenylphosphine (3 mmol/g; 83 mg) was stirred in dichloromethane (10 ml) for 30 min. tert-Butyl [1-(4-chlorobenzyl)piperidin-4-yl]{(2S)-3-[5-fluoro-2-(hydroxymethyl)phenoxy]-2-hydroxy-2-methylpropyl}carbamate (134 mg) was added, followed by tetrachloromethane (100 μl), and the mixture was stirred at room temperature for 18 h. Additional portions of tetrachloromethane (2 ml) and polymer-bound triphenylphosphine (3 mmol/g; 166 mg) were added, and stirring was continued for 7 h. then the insoluble material was removed by filtration, and the solvent removed in vacuo to afford a brownish oil, which was used without purification in the next step.

APCI-MS: m/z 555 (MH⁺).

### Step IV

### (2-{[(2S)-3-{[1-(4-chlorobenzyl)piperidin-4-yl]amino}-2-hydroxy-2-methylpropyl]oxy}-4-fluorophenyl)methanesulfonic acid

Sodium sulphite (1.0 g) was suspended in water (2 ml). A solution of tert-butyl [1-(4-chlorobenzyl)piperidin-4-yl]{(2S)-3-[2-(chloromethyl)-5-fluorophenoxy]-2-hydroxy-2-methylpropyl}carbamate (0.25 mmol) in ethanol (4 ml) was added. The mixture was stirred overnight at 80° C. The intermediate (2-{[(2S)-3-{(tert-butoxycarbonyl)[1-(4-chlorobenzyl)piperidin-4-yl]amino}-2-hydroxy-2-methylpropyl]oxy}-4-fluorophenyl)methanesulfonic acid was purified by HPLC (water/acetonitrile), and the solvent was removed by freeze-drying. The residue was redissolved in dichloromethane (10 ml), and TFA (95% in water, 5 ml) was added. After stirring at room temperature for 3 h the solvent was removed in vacuo, and the residue purified by HPLC to afford the title compound, 17 mg (9%).

¹H-NMR (CD₃OD, 400 MHz): δ 7.52 (s, 4H), 7.35 (dd, J=8.2, 7.0 Hz, 1H), 6.84 (d, J=10.6 Hz, 1H), 6.73 (td, J=8.4, 2.2 Hz, 1H), 4.34 (s, 2H), 4.24 (d, J=13.6 Hz, 1H), 4.10 (d, J=13.3 Hz, 1H), 4.07 (s, 2H), 3.93 (d, J=9.7 Hz, 1H), 3.61 (d, J=12.4 Hz, 2H), 3.47 (m, 1H), 3.11 (br.s, 1H), 3.04 (d, J=12.7 Hz, 1H), 2.38 (brs, 2H), 2.15 (br.s, 1H), 1.40 (s, 3H).

### Example 5

### Urea, N-5-chloro-(2-{(2S)-3-[1-{(4-chlorobenzyl)-4-piperidinyl}amino]-2-hydroxypropoxy}-4-hydroxyphenyl)-N′-cyclopropyl-

### Step I

### 5-Chloro-2,4-dihydroxybenzoic acid

To a solution of 4-chlororesorcinol (43.3 g) in water (250 ml), sodiumbicarbonate (180 g) was added in portions. The reaction was refluxed for 2 h, cooled to room temperature and concentrated hydrochloric acid (150 ml) was added dropwise (pH<1). The mixture was cooled on ice and the precipitate collected. The brown solid was washed with water (5×50 ml) and dried to air yielding 11.4 g of the subtitled compound as a brown solid.

¹H-NMR (dmso-d₆, 400 MHz): δ 11.21 (s, 1H), 7.67 (s, 1H), 6.50 (s, 1H).

### Step II

### Ethyl 5-chloro-2,4-dihydroxybenzoate

To a solution of 5-chloro-2,4-dihydroxybenzoic acid (20.9 g) thionyl chloride (50 ml) was added dropwise. The reaction was stirred at 80° C. for 18 h, after which the solvent was removed in vacuo. The residue was redissolved in EtOAc (300 ml) and washed with an aqueous solution of NaHCO₃ (10%; 100 ml). The organic phase was washed with water (3×100 ml), dried and removed in vacuo. The residue was purified by suspending in EtOAc (150 ml) and heptane (150 ml) and filtrating over silica (230-400 mesh). The filtrate was concentrated in vacuo and resuspended in EtOAc (35 ml) and heptane (250 ml). This suspension was filtrated over silica (230-400 mesh) and the filtrate collected and concentrated in vacuo, giving 14.0 g of the subtitled compound as a white solid.

¹H-NMR (acetone-d₆, 300 MHz): δ 10.87 (broad), 7.78 (s, 1), 6.58 (s, 1H); 4.40 (m, 2H), 1.39 (m, 3H).

### Step III

### Ethyl 5-chloro-2-hydroxy-4-[(4-methoxybenzyl)oxy]benzoate

To a solution of ethyl 5-chloro-2,4-dihydroxybenzoate (1.96 g) in aceton (50 ml) were added 4-methoxybenzylchloride (1.42 g) and K₂CO₃ (1.25 g). The reaction was heated to reflux for 18 h, after which the solvent was removed in vacuo. The residue was redissolved in EtOAc and washed with water. The organic solvent was removed in vacuo and the residue recrystallized from methanol and subsequently from ethanol, yielding 1.38 g of the subtitled compound as a white solid.

¹H-NMR (acetone-d₆, 400 MHz): δ 11.01 (broad), 7.81 (s, 1H), 7.46 (m, 2H), 6.98 (m, 2H), 6.76 (s, 1H), 4.40 (m, 2H), 3.82 (s, 3H), 1.39 (m, 3H)

### Step IV

### 5-Chloro-2hydroxy-4-[(4-methoxybenzyl)oxy]benzoic acid

To solution of ethyl 5-chloro-2-hydroxy-4-[(4-methoxybenzyl)oxy]benzoate (1.38 g) in ethanol (15 ml) 1 M NaOH aq (15 ml) was added. The reaction was heated to reflux for 1 h, diluted with water (100 ml) and the pH adjusted with 1 M HCl aq (15 ml) to acidic conditions. The precipitate was filtered, washed with water and dried in a vacuum oven yielding 1.09 g of the subtitled compound as a white solid.

¹H-NMR (dmso-d₆, 400 MHz): δ 7.73 (s, 1H), 7.39 (m, 2H), 6.97 (m, 2H), 6.82 (s, 1H), 3.77 (s, 3H).

### Step V

### 5-Chloro-2-hydroxy-4-[(4-methoxybenzyl)oxy]benzoyl azide

To a solution of 5-chloro-2-hydroxy-4[(4-methoxybenzyl)oxy]benzoic acid (154 mg) and triethylamine (1 eq) in DCM (3 ml) DPPA (3 eq) was added. The reaction was stirred for 48 h, after which the solvent was removed in vacuo. The residue was suspended in acetonitrile and the precipitate collected (96 mg of the subtitled compound). The filtrate was purified over HPLC (water/acetonitrile) yielding 30 mg of the subtitled compound as a white solid.

¹H-NMR (CDCl₃, 400 MHz): δ 10.97 (broad), 7.76 (s, 1H), 7.38 (m, 2H), 6.94 (m, 2H), 6.57 (s, 1H), 3.83 (s, 3H).

### Step V

### 5-Chloro-6-[(4-methoxybenzyl)oxy]-1,3-benzoxazol-2(3H)-one

A solution of 5-chloro-2-hydroxy-4-[(4-methoxybenzyl)oxy]benzoyl azide (30 mg) in toluene (2 ml) was stirred at 100° C. for 18 h. The precipitate was collected, yielding 19 mg of the subtitled compound.

¹H-NMR (dmso-d₆, 400 MHz): δ 7.40-7.38 (m, 3H), 7.16 (s, 1H), 6.95 (m, 2H), 3.76 (s, 3H).

### Step VI

### N-{5-Chloro-4-[(4-methoxybenzyl)oxy]-2-[(2S)-oxiran-2-ylmethoxy]phenyl-N′-cyclopropyl urea

A solution of 5-chloro-6-[(4-methoxybenzyl)oxy]-1,3-benzoxazol-2(3H)-one (1.62 g) in cyclopropylamine (10 ml) was stirred at room temperature for 2 h and at 50° C. for 2 h. The solvent was removed in vacuo and the residue redissolved in DMF (25 ml) to which S-(+)-glycidylnosylate (1.4 g) and cesium carbonate (2.6 g) were added. The reaction was stirred at room temperature for 18 h. EtOAc (300 ml) was added and the organic phase was extracted with water (3×100 ml). The organic phase was dried and the solvent removed in vacuo. The residue was washed with EtOAc (5×5 ml) and dried in a vacuum oven, yielding 1.60 g of the subtitled compound as a light brown solid.

¹H-NMR (CD₃OD, 400 MHz): δ 8.25 (s, 1H), 7.51 (broad), 7.36 (m, 2H), 6.90 (m, 2H), 6.55 (s, 1H), 4.31-4.27 (m, 1H), 3.83-3.97 (m, 1H), 3.82 (s, 3H), 3.32 (m, 1H), 2.92 (m, 1H), 2.75-2.72 (m, 1H), 2.59 (m, 1H), 0.86 (m, 2H), 0.68 (m, 2H).

### Step VII

A solution of N-{5-Chloro-4-[(4-methoxybenzyl)oxy]-2-[(2S)-oxiran-2-ylmethoxy]phenyl-N′-cyclopropyl urea (126 mg) and 1-(4-chlorobenzyl)piperidin-4-amine (68 mg) in ethanol (3 ml) was heated to 80° C. for 18 h. The solvent was removed in vacuo and the residue purified over HPLC (water/acetonitrile with 0.1% TFA), yielding 38 mg of the titled compound as a white solid.

¹H-NMR (acetone-d₆, 400 MHz): δ 8.31 (s, 1H), 7.61-7.46 (m, 4H), 6.87 (broad, 1H), 6.62 (s, 1H), 4.38-4.35 (m, 3H), 4.10-4.06 (m, 1H), 3.94-3.90 (m, 1H), 3.69-3.66 (m, 4H), 3.40-3.35 (m, 1H), 3.18 (m, 2H), 2.56-2.47 (m, 3H) 2.34-2.21 (m, 2H), 0.64-0.60 (m, 2H), 0.47-0.41 (m, 2H); APCI-MS: m/z 523 (MH⁺).

### Example 6

### Urea, N-(2-{(2S)-3-[1-{(4-chlorobenzyl)-4-piperidinyl}amino]-2-hydroxypropoxy}-phenyl)-N′-ethyl-

### Step I

### N-ethyl-N′-{2-[(2S)-oxiran-2-ylmethoxy]phenylurea

Two stock solutions of 0.2 M N-ethyl-N′-(2-hydroxyphenyl)urea in DMF and 0.2 M [(2S)-2methyloxiran-2-yl]methyl 3-nitrobenzenesulfonate in DMF were combined (total volume 100 μL). To this mixture cesium carbonate (0.03 mmol) was added and the reaction was stirred at room temperature for 18 h. The mixture was partitioned between water and DCM and the organic layer washed with water. The organic solvent was removed and the compound used without further purification in step II, example 6

APCI-MS: m/z 251 (MH⁺).

### Step II

Two stock solutions of 0.1 M N-ethyl-N′-{2-[(2S)-oxiran-2-ylmethoxy]phenylurea in EtOH and 0.1 M 1-(4-chlorobenzyl)piperidin-4-amine in EtOH were combined (total volume 400 μL). The mixture was heated to 80° C. for 12 h, after which the solvent was is removed yielding the titled compound.

APCI-MS: m/z 476 (MH⁺).

### Example 7

### (2S)-1-(2-ethylphenoxy)-3[(1-[4-chlorobenzyl]4-piperidinyl)amino]propan-2-ol

Prepared following procedure as described for example 6 using 2-ethyl phenol, S-glycidyl nosylate and 1-(4-chlorobenzyl)piperidin-4-amine as intermediates as an intermediate.

APCI-MS: m/z 403 (MH⁺).

### Example 8

### (2S)-1-[2-(-hydroxyethyl)phenoxy]-2-methyl-3[(1-[4-chlorobenzyl]-4-piperidinyl)amino]propan-2-ol trifluoroacetate salt

Prepared following procedure as described for example 6 using 2-ethoxyphenol, [(2S)-2-methyloxiran-2-yl]methyl-3-nitrobenzenesulfonate and 1-(4-chlorobenzyl)piperidin-4-amine as intermediates as an intermediate. APCI-MS: m/z 434 (MH⁺).

### Example 9

### 2-({2S}-3-[(1-[4-chlorobenzyl]-4-piperidinyl)amino]-2-hydroxy-2-methylpropoxy)benzaldehyde

Prepared following procedure as described for example 6 using 2-hydroxybenzaldehyde, [(2S)-2-methyloxiran-2-yl]methyl-3-nitrobenzenesulfonate and 1-(4-s chlorobenzyl)piperidin-4-amine as intermediates as an intermediate. APCI-MS: m/z 417 (MH⁺).

### Example 10

### 2-({2S}-3-[(1-[4-chlorobenzyl]-4-piperidinyl)amino]-2-hydroxypropoxy)-N-cyclopropylbenzamide

### Step I

### N-cyclopropyl-2-fluoro-6-hydroxy benzamide

To a suspension of PS-carbodiimid (1.09 mmol/g; 6 g) in chloroform (40 ml) and DMF (10 ml) first cyclopropylamine (315 mg) and consequently 2-fluoro-6-hydroxy benzoic acid (780 mg) were added. The reaction was stirred at room temperature for 48 h. The mixture was filtered and the solvent removed in vacuo. The residue was purified by flash chromatography (DCM/EtOH) yielding 243 mg of the subtitled compound.

APCI-MS: m/z 196 (MH⁺).

### Step II

### N-cyclopropyl-2-fluoro-6-[(2S)-oxiran-2ylmethoxy]benzamide

To a solution of N-cyclopropyl-2-fluoro-6-hydroxy benzamide (100 mg) and (S)-glycidyl nosylate (110 mg) in DMF (3 ml) cesium carbonate (250 mg) was added. The suspension was stirred at room temperature for 18 h. The mixture was partitioned between EtOAc and water and the organic layer was washed four times with water, dried over sodium sulphate and the solvent removed in vacuo yielding 89 mg of the subtitled compound. The material was used without further purification in step III of example 10.

APCI-MS: m/z 252 (MH⁺).

### Step III

Prepared following procedure as described for example 6, step II using N-cyclopropyl-2-fluoro-6-[(2S)-oxiran-2ylmethoxy]benzamide and 1-(4-chlorobenzyl)piperidin-4-amine as intermediates as an intermediate.

¹H-NMR (acetone-d₆, 400 MHz): δ 7.62 (m, 2H), 7.47 (m, 2H), 7.39-7.36 (m, 1H), 6.91 (m, 1H), 6.79 (m, 1H), 4.48 (s, 2H), 4.41-4.14 (m, 7H), 2.97-2.92 (m, 1H), 2.60-2.51 (m, 4H), 0.77-0.72 (m, 2H), 0.63-0.59 (m, 2H); APCI-MS: m/z 476 (MH⁺).

### Example 11

### Methyl 2-({2S}-3-[(1-[4-chlorobenzyl]-4-piperidinyl)amino]-2-hydroxypropoxy)-4-fluorobenzoate trifluoroacetic acid salt

Prepared following procedure as described for example 6 methyl 4-fluoro-2-hydroxybenzoate, S-glycidyl nosylate and 1-(4-chlorobenzyl)piperidin-4-amine as intermediates as an intermediate. APCI-MS: m/z 451 (MH⁺).

### Example 12

### N-[2-({2S}-3-[(1-[4-chlorobenzyl]-4-piperidinyl)amino]-2-hydroxypropoxy)-4-chlorophenyl acetamide

### Step I

### N-{4-chloro-2[(2S)-oxiran-2ylmethoxy]phenyl}acetamide

To a solution of N-(4-chloro-2-hydroxyphenyl)acetamide (1.27 g) and (2S)-glycidyl nosylate (0.93 g) in DMF (7.5 ml) cesium carbonate (2.6 g) was added and the reaction was stirred at room temperature for 24 h. The mixture was partioned between EtOAc (50 ml) and water (50 ml) and the organic phase was washed several times with water, dried over sodium sulphate and removed in vacuo. The residue was purified using flash chromatography (DCM/EtOH) to provide 317 mg of the subtitled compound.

¹H-NMR (CDCl₃, 400 MHz): δ 8.34 (m, 1H), 7.80 (broad), 7.00-6.98 (m, 1H), 6.90 (m, 1H), 4.40-4.36 (m, 1H), 3.95-3.91 (m, 1H), 3.41-3.39 (m, 1H), 2.99-2.97 (m, 1H), 2.80-2.78 (m, 1H), 2.22 (s, 3H); APCI-MS: m/z 242 (MH⁺).

### Step II

A mixture of N-{4-chloro-2-[(2S)-oxiran-2-ylmethoxy]phenyl}acetamide (38 mg) and 1-(4-chlorobenzyl)piperidin-4-amine (35 mg) in EtOH (3 ml) was stirred at 80° C. for 5 h. The solvent was removed in vacuo and the residue purified over HPLC (water/acetonitrile with 0.1% TFA) yielding 41 mg of the titled compound.

¹H-NMR (D2O, 400 MHz): δ 7.39-7.25 (m, 5H), 6.99-6.90 (m, 3H), 4.20-4.14 (m, 3H), 4.00-3.92 (m, 2H), 3.21-2.95 (m, 4H), 2.25 (m, 2H), 2.01 (s, 3H), 1.90-1.79 (m, 2H);

APCI-MS: m/z 466 (MH⁺).

The invention further relates to compounds selected from
Methyl (2-{[(2S)-3-{[1-(4-chlorobenzyl)piperidin-4-yl]amino}-2-hydroxypropyl]oxy}-4-fluorophenyl)propanoate;
N-[2-({2S}-3-[(1-[4-chlorobenzyl]-4-piperidinyl)amino]-2-hydroxypropoxy)-4-chlorophenyl acetamide;
(2-{[(2S)-3-{[1-(4-chlorobenzyl)piperidin-4-yl]amino}-2-hydroxy-2-methylpropyl]oxy}-4-fluorophenyl)methanesulfonic acid;
Urea, N-5-chloro-(2-{(2S)-3-[1-{(4-chlorobenzyl)-4-piperidinyl}amino]-2-hydroxypropoxy}-4-hydroxyphenyl)-N′-cyclopropyl-;
Urea, N-(2-{(2S)-3-[1-{(4-chlorobenzyl)-4-piperidinyl}amino]-2-hydroxypropoxy}-phenyl)-N′-ethyl-;
(2S)-1-(2-ethylphenoxy)-3[(1-[4-chlorobenzyl]4-piperidinyl)amino]propan-2-ol;
(2S)-1-[2-(-hydroxyethyl)phenoxy]-2-methyl-3[(1-[4-chlorobenzyl]-4-piperidinyl)amino]propan-2-ol;
2-({2S}-3-[(1-[4-chlorobenzyl]-4-piperidinyl)amino]-2-hydroxy-2-methylpropoxy)benzaldehyde;
2-({2S}-3-[(1-[4-chlorobenzyl]-4-piperidinyl)amino]-2-hydroxypropoxy)-N-cyclopropylbenzamide; and
Methyl 2-({2S}-3-[(1-[4-chlorobenzyl]-4-piperidinyl)amino]-2-hydroxypropoxy)-4-fluorobenzoate,
or pharmaceutical salts, solvates or solvates salts thereof.

### Example 13

### Inflammatory Cell Influx Experiment in LPS-Challenged Rats

The effect of CCR1 receptor antagonist 2-{2-chloro-5-{[(2S)-3-(5-chloro-1′H,3H-spiro[1-benzozuran-2,4′-piperidin]-1′-yl)-2-hydroxypropyl]oxy}-4-[(methylamino)carbonyl]phenoxy}-2-methylpropanoic acid referred to here as Compound A, and a muscarinic antagonist, tiotropium, referred to here as Compound B, and their combination, on inflammatory cell influx was examined by monitoring the effect on total and neutrophil cell number in bronchoalveolar lavage (BAL) fluid of rats challenged intra-tracheally (i.t.) with Lipopolysaccharide (LPS)N=10 [N=7 rats per treatment group]. Saline group n=2

### Methodology

Preparation of solutions: Both Compound A and Compound B were was dissolved in A Vehicle containing the following ingredients (mg/mL): Sodium chloride (8.5), EDTA (0.1), Citric acid dried (0.15), Sodium citrate (0.5), Polysorbat 80 (0.2) in Milli-Q water) to the final concentrations of 0.001 or 0.01 μg/ml (compound A) and 0.1 μg/ml (compound B).

Compound A/Compound B mixed formulations were made by mixing 0.002 or 0.02 μg/ml Compound A in Vehicle and 0.2 μg/ml Compound B in Vehicle to the final concentrations of 0.001/0.1 μg/mL Compound A/Compound B and 0.01/0.1 μg/mL Compound A/Compound B.

LPS (Lipopolysaccharide B. E. coli 026:B6) was dissolved in saline to a final concentration of 2.5 μg/ml

Treatments: Rats were anaesthetized with Isofluran and put in a supine position, head up, on a board tilted at 30°. Animals were intratracheally instilled with solutions (1 ml/kg) of Compound A/Compound B (0.001/0.1 μg/kg), Compound A/Compound B (0.01/0.1 μg/kg), Compound A (0.001 or 0.01 μg/kg) alone, Compound B (0.1 μg/kg) alone, or with Saline (negative and positive control animals). Rats remained in this position until regaining consciousness. The drugs were administrated 30 min before LPS instillation.

LPS instillation: Rats were anaesthetized with Isofluran and put in a supine position, head up, on a board tilted at 30°. LPS or saline alone (negative control) in a volume of 200 μl was administered i.t. using a modified metal cannula. Rats remained in this position until regaining consciousness.

Termination: 4 hours after the LPS challenge, rats were intraperitoneally injected with 2 mL of a mixture of pentobarbital (60 mg/ml, Apoteksbolaget, Sweden) and PBS (1:1) for 1-2 min.

Bronchoalveolar lavage (BAL): After termination, collection of BAL fluid was performed twice with PBS. The BAL fluid was centrifuged and the cell pellet was resuspended in PBS. The total numbers of BAL cells were counted in a SYSMEX cell counter.

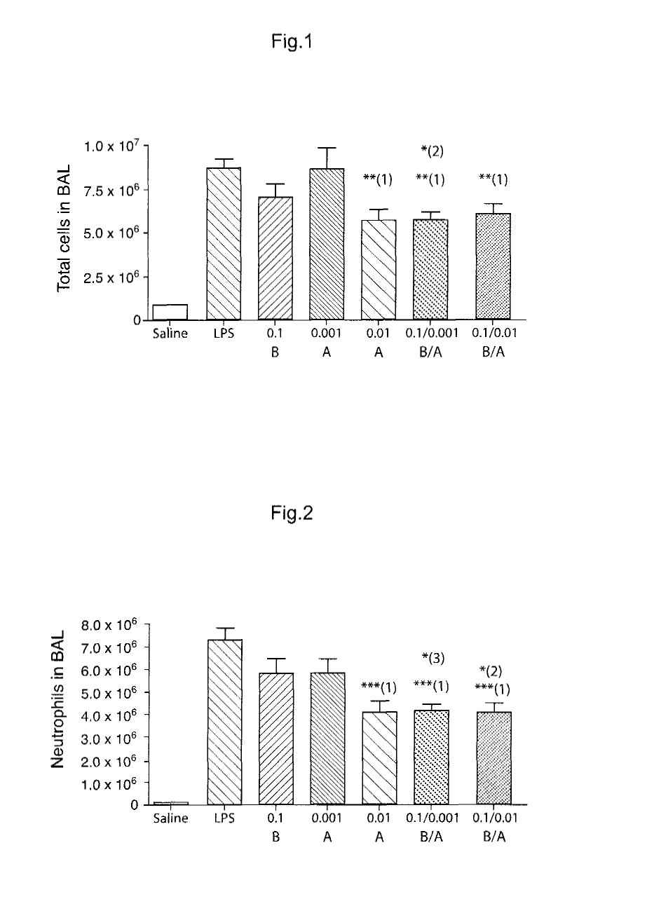

The results of the experiments are shown in FIGS. 1 and 2. In FIGS. 1 and 2 “saline” rats represents the negative control rats treated with Saline vehicle and challenged with saline. “Saline vehicle/LPS” animals represent the positive control rats treated with Saline vehicle and challenged with LPS. The remaining five groups were all treated with the specified drugs and challenged with LPS.

### Example 14

### Inflammatory Cell Influx Experiment in LPS-Challenged Rats

The effect of a CCR1 receptor antagonist, N-{5-Chloro-2-[((2S)-3-{[1-(4-chlorobenzyl)piperidin-4-yl]amino}-2-hydroxy-2-methylpropyl)oxy]-4-hydroxyphenyl}acetamide, referred to here as Compound C and a muscarinic antagonist, tiotropium, referred to here as Compound B, and their combination, on inflammatory cell influx was examined by monitoring the effect on total and neutrophil cell number in bronchoalveolar lavage (BAL) fluid of rats challenged intra-tracheally (i.t.) with Lipopolysaccharide (LPS)N=10 [N=10 rats per treatment group] Saline group n=2.

### Methodology

Preparation of solutions: Compound C was dissolved in in saline (0.9% NaCl) to the final concentrations of 30 μg/ml. Compound B was dissolved in vehicle to a final concentration of 0.1 μg/ml.

Compound C/Compound mixed formulations were made by mixing 60 μg/ml Compound C in saline (0.9% NaCl) and 0.2 μg/ml Compound B in vehicle to the final concentrations of 30/0.1 μg/mL Compound C/Compound B.

LPS (Lipopolysaccharide B. E. coli 026:B6) was dissolved in saline to a final concentration of 2.5 μg/ml

Treatments: Rats were anaesthetized with Isofluran and put in a supine position, head up, on a board tilted at 30°. Animals were intratracheally instilled with solutions (1 ml/kg) of Compound C/Compound B (30/0.1 μg/kg), Compound C (30 μg/kg) alone, Compound B (0.1 μg/kg) alone, or with Saline (negative and positive control animals). Rats remained in is this position until regaining consciousness. The drugs were administrated 30 min before LPS instillation.

LPS instillation: Rats were anaesthetized with Isofluran and put in a supine position, head up, on a board tilted at 30°. LPS or saline alone (negative control) in a volume of 200 μl was administered i.t. using a modified metal cannula. Rats remained in this position until regaining consciousness.

Termination: 4 hours after the LPS challenge, rats were intraperitoneally injected with 2 mL of a mixture of pentobarbital (60 mg/ml, Apoteksbolaget, Sweden) and PBS (1:1) for 1-2 min.

Bronchoalveolar lavage (BAL): After termination, collection of BAL fluid was performed twice with PBS. The BAL fluid was centrifuged and the cell pellet was resuspended in PBS. The total numbers of BAL cells were counted in a SYSMEX cell counter.

The results of the experiments are shown in FIGS. 3 and 4. In FIGS. 3 and 4 “saline” rats represents the negative control rats treated with Saline vehicle and challenged with saline. “Saline vehicle/LPS” animals represent the positive control rats treated with Saline vehicle and challenged with LPS. The remaining five groups were all treated with the specified drugs and challenged with LPS.

The results from these in vivo studies clearly show that the combination of two non-effective dosages of a CCR1 antagonist and a muscarinic antagonist leads to a significant reduction in the influx of pro-inflammatory cells, like neutrophils, in the bronchial lavage fluid, over both the LPS challenged group, as well as those groups treated with a CCR1 antagonist or a muscarinic antagonist. It may be concluded that such a combined treatment will lead to an improved control over the inflammatory element of the pathology of airway diseases, like COPD. The lowering in inflammatory cell levels following this combination is treatment will lead to a decrease in excessarbations of disease, i.e. a reduction in bronchoconstriction and a diminished mucus production and plugging. In addition, it is contemplated that patients that receive a combination treatment containing both a muscarinic receptor and a CCR1 receptor antagonist will need to be treated with less amount of both active ingredients, compared to patients that are on single treatment.

1. A pharmaceutical product comprising, in combination,
(a1) a first active ingredient, which is a compound of general formula 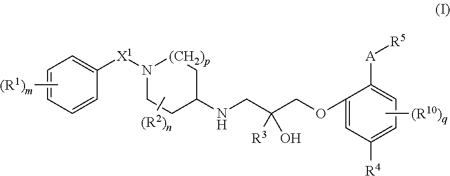
wherein:
m is 0, 1 or 2;
R¹ is halogen, C₁₋₃haloalkyl or cyano;
X¹ is —CH₂— or —C(O)—;
n is 0, 1 or 2;
p is 0, 1 or 2;
R² is C₁₋₆cycloalkyl; or
R² forms a bicyclic ring together with the ring it is attached to;
R³ is hydrogen or C₁₋₄alkyl;
R⁴ is hydrogen, halogen, hydroxyl, C₁₋₆hydroxyalkyl, optionally substituted by one substituent independently selected from halogen, cyano, amino (—NH₂), amido (—CONH₂), hydroxyl, oxo (═O), C₁₋₆haloalkyl, carboxyl, C₁₋₆alkoxy, C₁₋₆alkoxycarbonyl, C₁₋₆alkylcarbonylamino and a 3 to 6-membered saturated or unsaturated ring, optionally comprising one or more heteroatom selected from nitrogen, oxygen and sulphur, and optionally further comprising a bridging group, the ring being optionally substituted with one or more substituent independently selected from halogen, hydroxyl, oxo (═O), C₁₋₆alkyl, C₁₋₆hydroxyalkyl and C₁₋₆haloalkyl;
A is a bond or C₁₋₆haloalkyl;
R⁵ is hydrogen, hydroxyl, —NHC(O)R⁶, —NHS(O)₂R⁶, —C(O)NR⁷R⁸, —COOR⁹ or SO₃R⁹;
R⁶ is hydrogen, C₁₋₆alkyl or a 3 to 6-membered saturated or unsaturated ring, optionally comprising one or more heteroatom selected from nitrogen, oxygen and sulphur, and optionally further comprising a bridging group, the ring being optionally substituted with one or more substituent independently selected from halogen, hydroxyl, C₁₋₆alkyl, C₁₋₆ hydroxyalkyl and C₁₋₆haloalkyl, oxo (═O) and —OR⁹;
R⁷ and R⁸ each independently represent (i) hydrogen atom,
(ii) a 3 to 6-membered saturated or unsaturated ring, optionally comprising one or more heteroatom selected from nitrogen, oxygen and sulphur, and optionally further comprising a bridging group, the ring being optionally substituted with one or more substituent independently selected from halogen, hydroxyl, oxo (═O), C₁₋₆alkyl, C₁₋₆hydroxyalkyl and C₁₋₆haloalkyl,
(iii) a C₁₋₆alkyl group, optionally substituted by one or more substituent independently selected from halogen, amino (—NH₂), hydroxyl, oxo (═O), C₁₋₆haloalkyl, carboxyl, C₁₋₆alkoxy, C₁₋₆alkoxycarbonyl, C₁₋₆alkylcarbonylamino and a 3 to 6-membered saturated or unsaturated ring, optionally comprising one or more heteroatom selected from nitrogen, oxygen and sulphur, and optionally further comprising a bridging group, the ring being optionally substituted with one or more substituent independently selected from halogen, hydroxyl, oxo (═O), C₁₋₆alkyl, C₁₋₆hydroxyalkyl and C₁₋₆haloalkyl, or
(iv) C₁₋₆alkylsulphonyl, or
(v) R⁷ and R⁸ together with the nitrogen atom to which they are attached form a 4 to 7-membered saturated heterocyclic ring that optionally further comprises a ring nitrogen, oxygen or sulphur atom and that is optionally fused to a benzene ring to form a 8 to 11-membered ring system, the heterocyclic ring or ring system being optionally substituted with one or more substituent independently selected from halogen, hydroxyl, amido (—CONH₂), C₁₋₆allyl, C₁₋₆hydroxyalkyl, C₁₋₆alkoxy, C₁₋₆alkoxycarbonyl, C₁₋₆haloalkyl, C₁₋₆alkylamino, di-C₁₋₆alkylamino, C₁₋₆alkylcarbonyl, C₁₋₆alkylcarbonylamino, C₁₋₆alkylaminocarbonyl, di-C₁₋₆alkylaminocarbonyl, phenyl, halophenyl and phenylcarbonyl;
R⁹ is hydrogen or C₁₋₆alkyl;
q is 0, 1 or 2;
R¹⁰ is halogen, hydroxyl, cyano, C₁₋₃haloalkyl or C₁₋₆alkoxy;
or a pharmaceutically acceptable salt thereof;
or,
(a2) a first active ingredient, which is a compound of general formula 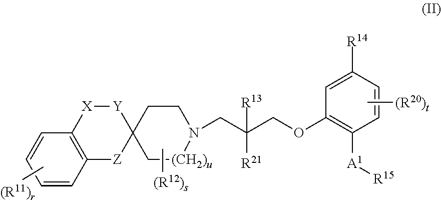
wherein:
r is 0, 1 or 2;
R¹¹ is halogen, cyano or C₁₋₆haloalkyl;
X, Y and Z is a bond, —O—, —NH—, CH₂— or —C(O)—, provided that only one of X, Y and Z is a bond, and provided that X and Y are not simultaneously —O— or —C(O)—;
s is 0, 1 or 2;
R¹² is C₁₋₆cycloalkyl;
u is 0 or 1;
R²¹ is hydrogen, hydroxyl or NH₂;
R¹³ is hydrogen or C₁₋₆alkyl;
A¹ is a bond or C₁₋₃alkyl;
R¹⁵ is hydrogen, hydroxyl, —NHC(O)R¹⁶, —NHS(O)₂R¹⁶, —C(O)NR¹⁷R¹⁸, —COOR¹⁹ or SO₃R¹⁹;
R¹⁴ is hydrogen, halogen, hydroxyl, OC(CH₃)₂COOH, C₁₋₆hydroxyalkyl optionally substituted by one or more substituent independently selected from halogen, cyano, amino (—NH₂), amido (—CONH₂), hydroxyl, oxo (═O), C₁₋₆haloalkyl, carboxyl, C₁₋₆alkoxy, C₁₋₆alkoxycarbonyl, C₁₋₆alkylcarbonylamino and a 3 to 6-membered saturated or unsaturated ring, optionally comprising one or more heteroatom independently selected from nitrogen, oxygen and sulphur, and optionally further comprising a bridging group, the ring being optionally substituted with one or more substituent independently selected from halogen, hydroxyl, oxo (═O), C₁₋₆alkyl, C₁₋₆hydroxyalkyl and C₁₋₆haloalkyl;
t is 0, 1 or 2;
R¹⁶ is hydrogen, C₁₋₃alkyl, NR¹⁷R¹⁸ or OR¹⁹;
R¹⁷ and R¹⁸ are independently selected from hydrogen, C₁₋₆alkyl and C₃₋₇cycloalkyl, or
R¹⁷ and R¹⁸ together with the nitrogen atom to which they are attached form a 4 to 7-membered heterocyclic ring, which is optionally substituted with on or more hydroxyl groups;
R¹⁹ is a hydrogen or C₁₋₃alkyl group; and
R²⁰ is halogen, cyano, C₁₋₃alkoxy or C₁₋₃haloalkyl,
or a pharmaceutically acceptable salt thereof;
and
(b) a second active ingredient, which is a muscarinic antagonist, or a pharmaceutically acceptable salt thereof,
provided the muscarinic antagonists is not selected from
a [2-((S)-Cyclohexyl-hydroxy-phenyl-methyl)-oxazol-5-ylmethyl]-dimethyl-(3-phenoxy-propyl)-ammonium salt,
a [2-((R)-Cyclohexyl-hydroxy-phenyl-methyl)-oxazol-5-ylmethyl]-dimethyl-(3-phenoxy-propyl)-ammonium salt,
a [2-((R)-Cyclohexyl-hydroxy-phenyl-methyl)-oxazol-5-ylmethyl]-dimethyl-(2-phenethyloxy-ethyl)-ammonium salt,
a [2-((R)-Cyclohexyl-hydroxy-phenyl-methyl)-oxazol-5-ylmethyl]-[3-(3,4-dichloro-phenoxy)-propyl]-dimethyl-ammonium salt,
a [2-((R)-Cyclohexyl-hydroxy-phenyl-methyl)-oxazol-5-ylmethyl]-[2-(3,4-dichloro-benzyloxy)-ethyl]-dimethyl-ammonium salt, or
a [2-(4-Chloro-benzyloxy)-ethyl]-[2-((R)-Cyclohexyl-hydroxy-phenyl-methyl)-oxazol-5-ylmethyl]-dimethyl-ammonium salt.

2. The pharmaceutical product according to claim 1 comprising, in combination,
(a1) a first active ingredient, which is a compound of general formula 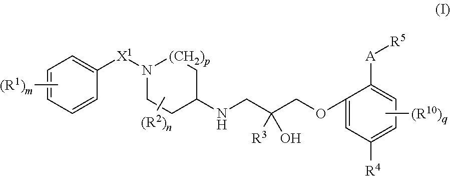
wherein:
m is 0 or 1; R¹ is halogen; X¹ is —CH₂—; n is 0; p is 0 or 1; R³ is C₁₋₄alkyl; R⁴ is hydroxyl; A is a bond; R⁵ is —NHC(O)R⁶; R⁶ is C₁₋₆alkyl and q is 0; or a pharmaceutically acceptable salt thereof;
or,
(a2) a first active ingredient, which is a compound of general formula 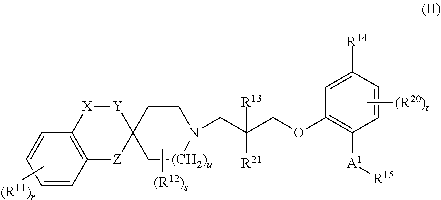
wherein:
r is 0 or 1; R¹¹ is halogen; X, Y and Z is a bond, —O— or CH₂—, provided that only one of X, Y and Z is a bond, and provided that X and Y are not simultaneously —O—; s is 0; u is 0 or 1; R²¹ is hydroxyl; R¹³ is hydrogen; A¹ is a bond; R¹⁵ is —C(O)NR¹⁷R¹⁸; R¹⁴ is OC(CH₃)₂COOH; t is 0 or 1; R¹⁷ and R¹⁸ are independently selected from hydrogen and C₁₋₆alkyl; and R²⁰ is halogen, or a pharmaceutically acceptable salt thereof;
and
(b) a second active ingredient, which is a muscarinic antagonist, or a pharmaceutically acceptable salt thereof,
provided the muscarinic antagonists is not selected from
a [2-((S)-Cyclohexyl-hydroxy-phenyl-methyl)-oxazol-5-ylmethyl]-dimethyl-(3-phenoxy-propyl)-ammonium salt,
a [2-((R)-Cyclohexyl-hydroxy-phenyl-methyl)-oxazol-5-ylmethyl]-dimethyl-(3-phenoxy-propyl)-ammonium salt,
a [2-((R)-Cyclohexyl-hydroxy-phenyl-methyl)-oxazol-5-ylmethyl]-dimethyl-(2-phenethyloxy-ethyl)-ammonium salt,
a [2-((R)-Cyclohexyl-hydroxy-phenyl-methyl)-oxazol-5-ylmethyl]-[3-(3,4-dichloro-phenoxy)-propyl]-dimethyl-ammonium salt,
a [2-((R)-Cyclohexyl-hydroxy-phenyl-methyl)-oxazol-5-ylmethyl]-[2-(3,4-dichloro-benzyloxy)-ethyl]-dimethyl-ammonium salt, or
a [2-(4-Chloro-benzyloxy)-ethyl]-[2-((R)-Cyclohexyl-hydroxy-phenyl-methyl)-oxazol-5-ylmethyl]-dimethyl-ammonium salt.

3. The pharmaceutical product according to claim 1, wherein the first active ingredient is selected from
N-(2{(2S)-3 [{(3R)-1-[(4-chlorophenyl)methyl]-3-pyrrolidinyl}amino]-2-hydroxypropoxy}-4-fluorophenyl)acetamide;
N-(2{(2S)-3[{(3S)-1-[(4-chlorophenyl)methyl]-3-pyrrolidinyl}amino]-2-hydroxypropoxy}-4-fluorophenyl)acetamide;
N-(2-{(2S)-3-[1-{(4-chlorobenzoyl)-4-piperidinyl}amino]-2-hydroxypropoxy}-4-hydroxyphenyl)acetamide;
(2-{[(2S)-3-{[(2R,5S)-1-(4-chlorobenzyl)-2,5-dimethylpiperidin-4-yl]amino}-2-hydroxy-2-methylpropyl]oxy}-4-fluorophenyl)acetic acid;
(2-{[(2S)-3-{[(3S,4R)-1-(4-chlorobenzyl)-3-methylpiperidin-4-yl]amino}-2-hydroxy-2-methylpropyl]oxy}-4-fluorophenyl)acetic acid;
(2-{[(2S)-3-{[(3R,4R)-1-(4-chlorobenzyl)-3-methylpiperidin-4-yl]amino}-2-hydroxy-2-methylpropyl]oxy}-4-fluorophenyl)acetic acid;
(2-{[(2S)-3-{[(2R,4S,5S)-1-(4-chlorobenzyl)-2,5-dimethylpiperidin-4-yl]amino}-2-hydroxy-2-methylpropyl]oxy}-4-fluorophenyl)acetic acid;
(2-{[(2S)-3-{[(2R,4R,5S)-1-(4-chlorobenzyl)-2,5-dimethylpiperidin-4-yl]amino}-2-hydroxy-2-methylpropyl]oxy}-4-fluorophenyl)acetic acid;
(2-{[(2S)-3-{[(2S,4R,5R)-1-(4-chlorobenzyl)-2,5-dimethylpiperidin-4-yl]amino}-2-hydroxy-2-methylpropyl]oxy}-4-fluorophenyl)acetic acid;
(2-{[(2S)-3-{[(2S,4S,5R)-1-(4-chlorobenzyl)-2,5-dimethylpiperidin-4-yl]amino}-2-hydroxy-2-methylpropyl]oxy}-4-fluorophenyl)acetic acid;
Methyl (2-{[(2S)-3-{[1-(4-chlorobenzyl)piperidin-4-yl]amino}-2-hydroxypropyl]oxy}-4-fluorophenyl)propanoate;
N-[2-({2S}-3-[(1-[4-chlorobenzyl]-4-piperidinyl)amino]-2-hydroxypropoxy)-4-chlorophenyl acetamide;
N-[2-({2S}-3-[(1-[4-chlorobenzyl]-4-piperidinyl)amino]-2-hydroxy-2-methylpropoxy)-4-hydroxyphenyl]acetamide;
N-[2-({2S}-3-[(1-[4-chlorobenzyl]-4-piperidinyl)amino]-2-hydroxy-2-methylpropoxy)-4-fluorophenyl]acetamide;
N-[5-chloro-[2-({2S}-3-[(1-[4-chlorobenzyl]-4-piperidinyl)amino]-2-hydroxy-2-methylpropoxy)-4-hydroxyphenyl]acetamide;
N-[5-chloro-[2-({2S}-3-[(1-[4-chlorobenzyl]-4-piperidinyl)amino]-2-hydroxy-2-methylpropoxy)-4-hydroxyphenyl]propaneamide;
(2-{[(2S)-3-{[1-(4-chlorobenzyl)piperidin-4-yl]amino}-2-hydroxy-2-methylpropyl]oxy}-4-fluorophenyl)methanesulfonic acid;
Urea, N-5-chloro-(2-{(2S)-3-[1-{(4-chlorobenzyl)-4-piperidinyl}amino]-2-hydroxypropoxy}-4-hydroxyphenyl)-N′-cyclopropyl-;
Urea, N-(2-{(2S)-3-[1-{(4-chlorobenzyl)-4-piperidinyl}amino]-2-hydroxypropoxy}-phenyl)-N′-ethyl-;
(2S)-1-(2-ethylphenoxy)-3[(1-[4-chlorobenzyl]-4-piperidinyl)amino]propan-2-ol;
(2S)-1-[2-(-hydroxyethyl)phenoxy]-2-methyl-3[(1-[4-chlorobenzyl]-4-piperidinyl)amino]propan-2-ol;
2-({2S}-3-[(1-[4-chlorobenzyl]-4-piperidinyl)amino]-2-hydroxy-2-methylpropoxy)benzaldehyde;
2-({2S}-3-[(1-[4-chlorobenzyl]-4-piperidinyl)amino]-2-hydroxypropoxy)-N-cyclopropylbenzamide;
Methyl 2-({2S}-3-[(1-[4-chlorobenzyl]-4-piperidinyl)amino]-2-hydroxypropoxy)-4-fluorobenzoate;
or a pharmaceutically acceptable salt thereof.

4. The pharmaceutical product according to claim 1, wherein the first active ingredient is selected from
N-(2-{[(2S)-3-(5-chloro-1′H,3H-spiro[1-benzofuran-2,4′-piperidin]-1′-yl)-2-hydroxypropyl]oxy}-4-hydroxyphenyl)acetamide;
N-(2-{[(2S)-3-(5-chloro-1′H-spiro[1,3-benzodioxole-2,4′-piperidin]-1′-yl)-2-hydroxypropyl]oxy}-4-hydroxyphenyl)acetamide trifluoroacetate (salt);
2-{[(2S)-3-(5-chloro-1′H,3H-spiro[1-benzofuran-2,4′-piperidin]-1′-yl)-2-hydroxypropyl]oxy}-4-hydroxy-N-methylbenzamide trifluoroacetate (salt);
2-{[(2S)-3-(5-chloro-1′H,3H-spiro[1-benzofuran-2,4′-piperidin]-1′-yl)-2-hydroxypropyl]oxy}-4-hydroxybenzoic acid trifluoroacetate (salt);
N-(2-{[(2S)-3-(5-chloro-1′H,3H-spiro[2-benzofuran-1,4′-piperidin]-1′-yl)-2-hydroxypropyl]oxy}-4-hydroxyphenyl)acetamide trifluoroacetate (salt);
2-{[(2S)-3-(5-chloro-1′H,3H-spiro[2-benzofuran-1,4′-piperidin]-1′-yl)-2-hydroxypropyl]oxy}-4-hydroxy-N-methylbenzamide;
N-(2-{[(2S)-3-(5-fluoro-1′H,3H-spiro[1-benzofuran-2,4′-piperidin]-1′-yl)-2-hydroxypropyl]oxy}-4-hydroxyphenyl)acetamide;
2-{[(2S)-3-(5-fluoro-1′H,3H-spiro[1-benzofuran-2,4′-piperidin]-1′-yl)-2-hydroxypropyl]oxy}-4-hydroxy-N-methylbenzamide;
N-[2-({(2S)-3-[(2R)-5-chloro-1′H,3H-spiro[1-benzofuran-2,3′-pyrrolidin]-1′-yl]-2-hydroxypropyl}oxy)-4-hydroxyphenyl]acetamide;
N-(2-{[(2S)-3-(5-chloro-1′H,3H-spiro[1-benzofuran-2,4′-piperidin]-1′-yl)-2-hydroxypropyl]oxy}-4-hydroxyphenyl)urea trifluoroacetate (salt);
4-fluoro-2-{[(2S)-3-(5-fluoro-1′H,3H-spiro[1-benzofuran-2,4′-piperidin]-1′-yl)-2-hydroxypropyl]oxy}benzoic acid hydrochloride;
N-(2-{[(2S)-3-(5-chloro-1′H,3H-spiro[1-benzofuran-2,4′-piperidin]-1′-yl)-2-hydroxypropyl]oxy}-4-fluorophenyl)urea trifluoroacetate (salt);
N-(2-{[(2S)-2-amino-3-(5-fluoro-1′H,3H-spiro[1-benzofuran-2,4′-piperidin]-1′-yl)propyl]oxy}-4-hydroxyphenyl)acetamide bis(trifluoroacetate) (salt);
Benzaldehyde, 2-[(2S)-3-(5-chlorospiro[benzofuran-2(3H), 4′-piperidin]-1′-yl)-2-hydroxypropoxy]-;
Spiro[benzofuran-2(3H), 4′-piperidine]-1′-ethanol, 5-chloro-α-[[2-(2-hydroxyethyl)phenoxy]methyl]-, (αS)-;
Spiro[benzofuran-2(3H),4′-piperidine]-1′-ethanol, 5-chloro-α-[[2-(hydroxymethyl)phenoxy]methyl]-, (αS)-;
N-(2-{[(2S)-3-(5-chloro-1′H,3H-spiro[1-benzofuran-2,4′-piperidin]-1′-yl)-2-hydroxypropyl]oxy}-5-chloro-4-hydroxyphenyl)acetamide;
2-Chloro-5-{[(2S)-3-(5-chloro-1′H,3H-spiro[1-benzofuran-2,4′-piperidin]-1′-yl)-2-hydroxypropyl]oxy}-(4-{acetylamino}phenoxy)acetic acid;
5-{[(2S)-3-(5-Chloro-1′H,3H-spiro[1-benzofuran-2,4′-piperidin]-1′-yl)-2-hydroxypropyl]oxy}-(4-{acetylamino}phenoxy)acetic acid;
{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}acetic acid;
2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid;
(2-Chloro-5-{[(2S)-3-(5-chloro-1′H,3H-spiro[1-benzofuran-2,4′-piperidin]-1′-yl)-2-hydroxypropyl]oxy}-4-{[(3S)-3-hydroxypyrrolidin-1-yl]carbonyl}phenoxy)acetic acid;
5-Chloro-2-{[(2S)-3-(5-chloro-1′H,3H-spiro[1-benzofuran-2,4′-piperidin]-1′-yl)-2-hydroxypropyl]oxy}-4-(cyanomethoxy)benzoic acid trifluoroacetate (salt);
2-{[(2S)-3-(5-chloro-1′H,3H-spiro[1-benzofuran-2,4′-piperidin]-1′-yl)-2-hydroxypropyl]oxy}-5-chloro-4-(2,2-difluoroethoxy)benzoic acid trifluoroacetate (salt);
5-Chloro-2-{[(2S)-3-(5-chloro-1′H,3H-spiro[1-benzofuran-2,4′-piperidin]-1′-yl)-2-hydroxypropyl]oxy}-4-(3,3,3-trifluoropropoxy)benzoic acid trifluoroacetate (salt);
N-(2-{3-[5-chloro-1′H,3H-spiro[1-benzofuran-2,4′-piperidin]-1′-yl]propoxy}phenyl)acetamide trifluoroacetate (salt);
Methyl 3-(2-{[(2S)-3-(5-chloro-1′H3H-spiro[1-benzofuran-2,4′-piperidin]-1′-yl)-2-hydroxypropyl]oxy}-4-fluorophenyl)propanoic acid trifluo acetic acid salt;
N-(2-{[(2S)-3-({-spiro[indole-2-4′-piperidin]-3 (1H)-one}-1′-yl)-2-hydroxypropyl]oxy}-4-hydroxyphenyl)acetamide; and
(2-{[(2S)-3-(5-Chloro-1′H,3H-spiro[1-benzofuran-2,4′-piperidin]-1′-yl)-2-hydroxypropyl]oxy}-4-fluorophenyl)methanesulfonic acid,
or a pharmaceutically acceptable salt thereof.

5. The pharmaceutical product according to claim 1, wherein the second active ingredient is selected from: aclidinium bromide, glycopyrrolate, oxitropium bromide, pirenzepine, telenzepine, 3(R)-(2-hydroxy-2,2-dithien-2-ylacetoxy)-1-(3-phenoxypropyl)-1-azoniabicyclo [2.2.2]octane bromide, 3(R)-1-phenethyl-3-(9H-xanthene-9-carbonyloxy)-1-azoniabicyclo [2.2.2]octane bromide and (3R)-3-[(2S)-2-cyclopentyl-2-hydroxy-2-thien-2-ylacetoxy]-1-(2-phenoxyethyl)-1-azoniabicyclo [2.2.2]actane bromide.

6. The pharmaceutical product according to claim 1, wherein the muscarinic antagonist is tiotropium or a pharmaceutically acceptable salt thereof.

7. A pharmaceutical product comprising, in combination
(a) a first active ingredient, which is any one of N-{2-[((2S)-3-{[1-(4-chlorobenzyl)piperidin-4-yl]amino}-2-hydroxy-2-methylpropyl)oxy]-4-hydroxyphenyl}acetamide or N-{5-Chloro-2-[((2S)-3-{[1-(4-chlorobenzyl)piperidin-4-yl]amino}-2-hydroxy-2-methylpropyl)oxy]-4-hydroxyphenyl}acetamide or 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid or a pharmaceutically acceptable salt thereof, and
(b) a second active ingredient, which is tiotropium, or a pharmaceutically acceptable salt thereof.

8. The pharmaceutical product according to claim 1, wherein both active ingredients are in a composition suitable for inhalation.

9. The pharmaceutical product according to claim 1 for use in therapy.

10. (canceled)

11. (canceled)

12. A method of treating airway diseases, or chronic obstructive pulmonary disease or asthma, which comprises simultaneously, sequentially or separately administering:
(a) a (therapeutically effective) dose of a first active ingredient, which is a compound of formula (I) or (II), or a pharmaceutically acceptable salt thereof according to claim 1; and
(b) a (therapeutically effective) dose of a second active ingredient, which is a muscarinic antagonist, or a pharmaceutically acceptable salt thereof,
to a patient in need thereof, provided that the muscarinic antagonist is not selected from
a 2-((S)-Cyclohexyl-hydroxy-phenyl-methyl)-oxazol-5-ylmethyl]-dimethyl-(3-phenoxy-propyl)-ammonium salt,
a [2-((R)-Cyclohexyl-hydroxy-phenyl-methyl)-oxazol-5-ylmethyl]-dimethyl-(3-phenoxy-propyl)-ammonium salt,
a [2-((R)-Cyclohexyl-hydroxy-phenyl-methyl)-oxazol-5-ylmethyl]-dimethyl-(2-phenethyloxy-ethyl)-ammonium salt,
a [2-((R)-Cyclohexyl-hydroxy-phenyl-methyl)-oxazol-5-ylmethyl]-[3-(3,4-dichloro-phenoxy)-propyl]-dimethyl-ammonium salt,
a [2-((R)-Cyclohexyl-hydroxy-phenyl-methyl)-oxazol-5-ylmethyl]-[2-(3,4-dichloro-benzyloxy)-ethyl]-dimethyl-ammonium salt, or
a [2-(4-Chloro-benzyloxy)-ethyl]-[2-((R)-Cyclohexyl-hydroxy-phenyl-methyl)-oxazol-5-ylmethyl]-dimethyl-ammonium salt.

13. The method according to claim 12, wherein
(a) the first active ingredient is any one of N-{2-[((2S)-3-{[1-(4-chlorobenzyl)piperidin-4-yl]amino}-2-hydroxy-2-methylpropyl)oxy]-4-hydroxyphenyl}acetamide or N-{5-Chloro-2-[((2S)-3-{[1-(4-chlorobenzyl)piperidin-4-yl]amino}-2-hydroxy-2-methylpropyl)oxy]-4-hydroxyphenyl}acetamide or 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid or a pharmaceutically acceptable salt thereof, and
(b) the second active ingredient is tiotropium, or a pharmaceutically acceptable salt thereof.

14. Compounds selected from
Methyl (2-{[(2S)-3-{[1-(4-chlorobenzyl)piperidin-4-yl]amino}-2-hydroxypropyl]oxy}-4-fluorophenyl)propanoate;
N-[2-({2S}-3-[(1-[4-chlorobenzyl]-4-piperidinyl)amino]-2-hydroxypropoxy)-4-chlorophenyl acetamide;
(2-{[(2S)-3-{[1-(4-chlorobenzyl)piperidin-4-yl]amino}-2-hydroxy-2-methylpropyl]oxy}-4-fluorophenyl)methanesulfonic acid;
Urea, N-5-chloro-(2-{(2S)-3-[1-{(4-chlorobenzyl)-4-piperidinyl}amino]-2-hydroxypropoxy}-4-hydroxyphenyl)-N′-cyclopropyl-;
Urea, N-(2-{(2S)-3-[1-{(4-chlorobenzyl)-4-piperidinyl}amino]-2-hydroxypropoxy}-phenyl)-N′-ethyl-;
(2S)-1-(2-ethylphenoxy)-3[(1-[4-chlorobenzyl]-4-piperidinyl)amino]propan-2-ol;
(2S)-1-[2-(-hydroxyethyl)phenoxy]-2-methyl-3[(1-[4-chlorobenzyl]-4-piperidinyl)amino]propan-2-ol;
2-({2S}-3-[(1-[4-chlorobenzyl]-4-piperidinyl)amino]-2-hydroxy-2-methylpropoxy)benzaldehyde;
2-({2S}-3-[(1-[4-chlorobenzyl]-4-piperidinyl)amino]-2-hydroxypropoxy)-N-cyclopropylbenzamide; and
Methyl 2-({2S}-3-[(1-[4-chlorobenzyl]-4-piperidinyl)amino]-2-hydroxypropoxy)-4-fluorobenzoate,
or pharmaceutical salts, solvates or solvates salts thereof.

15. The method according to claim 12, wherein the airway disease is chronic obstructive pulmonary disease or asthma.
